(19) United States
(12) Patent Application Publication
(10) Pub. No.: US 2024/0189318 A1
(43) Pub. Date: Jun. 13, 2024

(54) THERAPEUTIC REGIMENS OF AN INHIBITOR OF THE ENZYMATIC ACTIVITY OF BRG1 AND BRM

(71) Applicant: FOGHORN THERAPEUTICS INC., Cambridge, MA (US)

(72) Inventor: Liyue HUANG, Sharon, MA (US)

(21) Appl. No.: 18/282,279

(22) PCT Filed: Mar. 18, 2022
(86) PCT No.: PCT/US2022/020943

§ 371 Date: Sep. 15, 2023

Related U.S. Application Data
(60) Provisional application No. 63/163,338, filed on Mar. 19, 2021.

(51) Int. Cl. A61K 31/5377; A61K 31/192; A61K 31/325; A61K 31/381; A61K 31/4196; A61K 31/4439; A61K 31/451; A61K 31/4525; A61K 31/4709; A61K 31/496; A61K 31/522; A61K 31/7048; A61P 35/00

(52) U.S. Cl. CPC A61K 31/5377; A61K 31/192; A61K 31/325; A61K 31/381; A61K 31/4196; A61K 31/4439; A61K 31/451; A61K 31/4525; A61K 31/4709; A61K 31/496; A61K 31/522; A61K 31/7048; A61P 35/00

(57) ABSTRACT

Disclosed are methods of administering the compound of formula (I) in a therapeutic regimen to a subject in need thereof, e.g., for treating cancer.

### BACKGROUND

The invention relates to methods utilizing an inhibitor of the enzymatic activity of BRG1 (Brahma-related gene-1) and BRM (Brahma) in a therapeutic regimen, e.g., for treating cancer use in a subject.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 and BRM. The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY OF THE INVENTION

The present invention features methods of administering the compound of formula (I) in a therapeutic regimen to a subject in need thereof, e.g., for treating cancer.

In one aspect, the invention features a method of providing the compound of formula (I) to a subject in need thereof, the compound of formula (I) having the following structure:

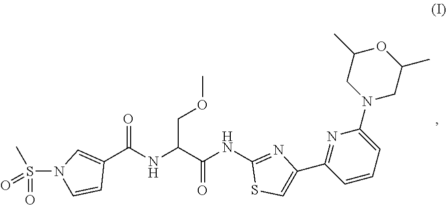 [SMILES: COCC(NC(=O)c1ccn(S(C)(=O)=O)c1)C(=O)Nc1nc(-c2cccc(N3CC(C)OC(C)C3)n2)cs1]

the method including the step of administering to the subject a therapeutic regimen providing the compound of formula (I), or a pharmaceutically acceptable salt thereof, to the subject; where the subject is not administered a CYP3A inhibitor, a CYP3A inducer, a sensitive CYP3A substrate with a narrow therapeutic index, a sensitive P-gp substrate with a narrow therapeutic index, a sensitive BCRP substrate with a narrow therapeutic index, or a combination thereof concomitantly with the regimen.

In another aspect, the invention provides a method of inhibiting cell proliferation in a cancer tissue (e.g., tissue containing cancer cells) in a subject in need thereof, the method including the step of contacting the cancer tissue with the compound of formula (I) according to a therapeutic regimen:

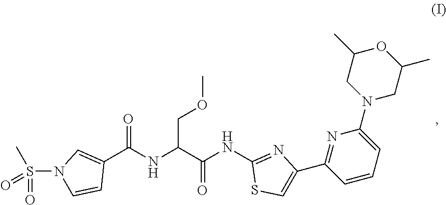 [SMILES: COCC(NC(=O)c1ccn(S(C)(=O)=O)c1)C(=O)Nc1nc(-c2cccc(N3CC(C)OC(C)C3)n2)cs1]

where the subject is not administered a CYP3A inhibitor, a CYP3A inducer, a sensitive CYP3A substrate with a narrow therapeutic index, a sensitive P-gp substrate with a narrow therapeutic index, a sensitive BCRP substrate with a narrow therapeutic index, or a combination thereof concomitantly with the regimen.

In some embodiments of either of the above aspects, the subject has cancer.

In another aspect, the invention provides a method of treating cancer in a subject in need thereof, the method including the step of administering to the subject a therapeutic regimen providing an effective amount of the compound of formula (I):

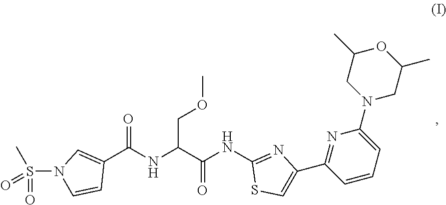 [SMILES: COCC(NC(=O)c1ccn(S(C)(=O)=O)c1)C(=O)Nc1nc(-c2cccc(N3CC(C)OC(C)C3)n2)cs1]

or a pharmaceutically acceptable salt thereof, where the subject is not administered a CYP3A inhibitor, a CYP3A inducer, a sensitive CYP3A substrate with a narrow therapeutic index, a sensitive P-gp substrate with a narrow therapeutic index, a sensitive BCRP substrate with a narrow therapeutic index, or a combination thereof concomitantly with the regimen.

In some embodiments of any of the above aspects, the cancer is non-small cell lung cancer, colorectal cancer, bladder cancer, cancer of unknown primary, glioma, breast cancer, melanoma, non-melanoma skin cancer, endometrial cancer, esophageal cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, penile cancer, bone cancer, or hematologic cancer.

In some embodiments, the cancer is esophageal cancer.

In some embodiments, the cancer is non-small cell lung cancer, colorectal cancer, bladder cancer, cancer of unknown primary, glioma, breast cancer, melanoma, non-melanoma skin cancer, endometrial cancer, penile cancer, bone cancer, renal cell carcinoma, prostate cancer, or hematologic cancer.

In some embodiments, the cancer is non-small cell lung cancer.

In some embodiments, the cancer is melanoma, prostate cancer, breast cancer, bone cancer, renal cell carcinoma, or hematologic cancer.

In some embodiments, the cancer is melanoma (e.g., uveal melanoma, mucosal melanoma, cutaneous melanoma).

In some embodiments, the melanoma is uveal melanoma (e.g., metastatic uveal melanoma, advanced uveal melanoma).

In some embodiments, the cancer is prostate cancer.

In some embodiments, the cancer is hematologic cancer (e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, non-Hodgkin's lymphoma).

In some embodiments, the hematologic cancer is acute myeloid leukemia.

In some embodiments, the cancer is breast cancer (e.g., ER positive breast cancer, an ER negative breast cancer, triple positive breast cancer, or triple negative breast cancer).

In some embodiments, the cancer is a bone cancer (e.g., Ewing's sarcoma).

In some embodiments, the cancer is a renal cell carcinoma (e.g., microphthalmia transcription factor family translocation renal cell carcinoma).

In some embodiments, the cancer is metastatic.

In some embodiments, the cancer is advanced.

In some embodiments, the cancer is resistant to, or failed to respond to prior treatment with, an anticancer therapy (e.g., a chemotherapeutic or cytotoxic agent, immunotherapy, surgery, radiotherapy, thermotherapy, or photocoagulation, or a combination thereof).

In some embodiments, the anticancer therapy is a chemotherapeutic or cytotoxic agent (e.g., a mitogen-activated protein kinase (MEK) inhibitor and/or a protein kinase C (PKC) inhibitor).

In some embodiments, the cancer is resistant to, or failed to respond to prior treatment with a PKC inhibitor.

In some embodiments, the method further features administering to the subject an anticancer therapy (e.g., a chemotherapeutic or cytotoxic agent, immunotherapy, surgery, radiotherapy, thermotherapy, photocoagulation, or a combination thereof).

In some embodiments, the anticancer therapy is surgery, a MEK inhibitor (e.g., selumetinib, binimetinib, or tametinib), or a PKC inhibitor (e.g., sotrastaurin or IDE196), or a combination thereof.

In some embodiments, the CYP3A inhibitor is a strong CYP3A inhibitor (e.g., boceprevir, cobicistat, danoprevir, elvitegravir, grapefruit juice, indinavir, itraconazole, ketoconazole, lopinavir, paritaprevir, ombitasvir, dasabuvir, posaconazole, ritonavir, saquinavir, telaprevir, tipranavir, telithromycin, troleandomycin, voriconazole, clarithromycin, idelalisib, nefazodone, nelfinavir, or a pharmaceutically acceptable salt thereof, or a combination thereof).

In some embodiments, the CYP3A inducer is a strong CYP3A inducer (e.g., apalutamide, carbamazepine, enzalutamide, mitotane, phenytoin, rifampin, St. John's wort, or a pharmaceutically acceptable salt thereof, or a combination thereof).

In some embodiments, the sensitive CYP3A substrate with a narrow therapeutic index is alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, tolvaptan, or a pharmaceutically acceptable salt thereof, or a combination thereof.

In some embodiments, the sensitive P-gp substrate with a narrow therapeutic index is an orally administered sensitive P-gp substrate with a narrow therapeutic index.

In some embodiments, the sensitive P-gp substrate with a narrow therapeutic index is dabigatran etexilate, digoxin, fexofenadine, loperamide, quinidine, talinolol, vinblastine, or a pharmaceutically acceptable salt thereof, or a combination thereof.

In some embodiments, the sensitive BCRP substrate with a narrow therapeutic index is an orally administered sensitive BCRP substrate with a narrow therapeutic index.

In some embodiments, the sensitive BCRP substrate with a narrow therapeutic index is coumestrol, daidzein, dantrolene, estrone-3-sulfate, genistein, prazosin, sulfasalazine, rosuvastatin, or a pharmaceutically acceptable salt thereof, or a combination thereof.

In some embodiments, the subject is not administered an acid-reducing agent (e.g., an antacid, H2 blocker (e.g., famotidine, cimetidine, ranitidine, nizatidine, or a combination thereof), proton pump inhibitor (e.g., omeprazole, lansoprazole, pantoprazole, rabeprazole, esomeprazole, dexlansoprazole, ilaprazole, or a combination thereof), or a combination thereof) concomitantly with the regimen; provided that, an acid-reducing agent that is an antacid may be concomitantly administered with the therapeutic regimen in a staggered dosing manner.

In some embodiments, the compound of formula (I) is administered orally.

In some embodiments, the compound of formula (I) is administered in a unit dosage form selected from the group consisting of capsule or tablet.

In some embodiments, the compound of formula (I) has the following structure:

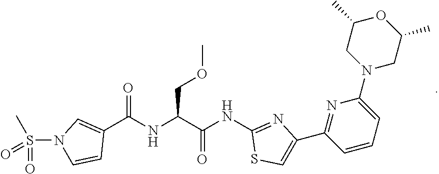 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

In some embodiments, the compound of formula (I) is administered as a pharmaceutical composition comprising the compound of formula (I) or a pharmaceutically acceptable salt thereof. In some embodiments, the pharmaceutical composition comprises one or more of a filler, a disintegrant, a wetting agent, a glidant, a lubricant, and a capsule shell. In some embodiments, the filler is microcrystalline cellulose, mannitol, or a combination thereof. In some embodiments, the pharmaceutical composition comprises 70 to 90% (w/w) of the filler. In some embodiments, the disintegrant is croscarmellose sodium. In some embodiments, the pharmaceutical composition comprises 4 to 6% (w/w) of the disintegrant. In some embodiments, the wetting agent is sodium lauryl sulfate. In some embodiments, the pharmaceutical composition comprises 0.5 to 1.5% (w/w) of the wetting agent. In some embodiments, the glidant is colloidal silicon dioxide. In some embodiments, the pharmaceutical composition comprises 1.5 to 2.5% (w/w) of the glidant. In some embodiments, the lubricant is magnesium stearate. In some embodiments, the pharmaceutical composition comprises 0.4 to 0.6% (w/w) of the lubricant. In some embodiments, the pharmaceutical composition comprises a capsule shell comprising a polymeric shell. In some embodiments, the polymeric shell comprises hypromellose and titanium dioxide. In some embodiments, the pharmaceutical composition is a unit dosage form. In some embodiments, the unit dosage form is a capsule. In some embodiments, the pharmaceutical composition comprises 2.5 to 20% (w/w) of the compound of formula (I) ora pharmaceutically acceptable salt thereof.

In some embodiments, the pharmaceutical composition comprises 2.5 mg to 20 mg of the compound of formula (I) or a pharmaceutically acceptable salt thereof.

In an aspect, the invention provides a pharmaceutical composition comprising the compound of formula (I) or a pharmaceutically acceptable salt thereof. In some embodiments, the pharmaceutical composition comprises one or more of a filler, a disintegrant, a wetting agent, a glidant, a lubricant, and a capsule shell. In some embodiments, the filler is microcrystalline cellulose, mannitol, or a combination thereof. In some embodiments, the pharmaceutical composition comprises 70 to 90% (w/w) of the filler. In some embodiments, the disintegrant is croscarmellose sodium. In some embodiments, the pharmaceutical composition comprises 4 to 6% (w/w) of the disintegrant. In some embodiments, the wetting agent is sodium lauryl sulfate. In some embodiments, the pharmaceutical composition comprises 0.5 to 1.5% (w/w) of the wetting agent. In some embodiments, the glidant is colloidal silicon dioxide. In some embodiments, the pharmaceutical composition comprises 1.5 to 2.5% (w/w) of the glidant. In some embodiments, the lubricant is magnesium stearate. In some embodiments, the pharmaceutical composition comprises 0.4 to 0.6% (w/w) of the lubricant. In some embodiments, the pharmaceutical composition comprises a capsule shell comprising a polymeric shell. In some embodiments, the polymeric shell comprises hypromellose and titanium dioxide. In some embodiments, the pharmaceutical composition is a unit dosage form. In some embodiments, the unit dosage form is a capsule. In some embodiments, the pharmaceutical composition comprises 2.5 to 20% (w/w) of the compound of formula (I) or a pharmaceutically acceptable salt thereof. In some embodiments, the pharmaceutical composition comprises 2.5 mg to 20 mg of the compound of formula (I) or a pharmaceutically acceptable salt thereof.

In some embodiments, the pharmaceutical composition comprises:
2.5 to 20% (w/w) of the compound of formula (I) or a pharmaceutically acceptable salt thereof;
70 to 90% (w/w) of the filler;
4 to 6% (w/w) of the disintegrant;
0.5 to 1.5% (w/w) of the wetting agent;
1.5 to 2.5% (w/w) of the glidant; and
0.4 to 0.6% (w/w) of the lubricant.

In some embodiments, the pharmaceutical composition comprises:
2.5 to 20% (w/w) of the compound of formula (I) or a pharmaceutically acceptable salt thereof;
70 to 90% (w/w) of microcrystalline cellulose, mannitol, or a combination thereof;
4 to 6% (w/w) of croscarmellose sodium;
0.5 to 1.5% (w/w) of sodium lauryl sulfate;
1.5 to 2.5% (w/w) of colloidal silicon dioxide; and
0.4 to 0.6% (w/w) of magnesium stearate.

In some embodiments, the pharmaceutical composition comprises 2.5 mg to 20 mg of the compound of formula (I) or a pharmaceutically acceptable salt thereof.

In some embodiments, the pharmaceutical composition comprises:
2.6% (w/w) of the compound of formula (I) or a pharmaceutically acceptable salt thereof;
50.8% (w/w) of microcrystalline cellulose;
38.1% (w/w) of mannitol;
5% (w/w) of croscarmellose sodium;
1.0% (w/w) of sodium lauryl sulfate;
2.0% (w/w) of colloidal silicon dioxide; and
0.5% (w/w) of magnesium stearate.

In some embodiments, the pharmaceutical composition comprises:
20% (w/w) of the compound of formula (I) or a pharmaceutically acceptable salt thereof;
48.2% (w/w) of microcrystalline cellulose;
37.0% (w/w) of mannitol;
5.8% (w/w) of croscarmellose sodium;
1.2% (w/w) of sodium lauryl sulfate;
2.3% (w/w) of colloidal silicon dioxide; and
0.6% (w/w) of magnesium stearate.

In some embodiments, the pharmaceutical composition comprises 2.5 mg of the compound of formula (I) or a pharmaceutically acceptable salt thereof. In some embodiments, the pharmaceutical composition comprises 20 mg of the compound of formula (I) or a pharmaceutically acceptable salt thereof. In some embodiments, the pharmaceutical composition is a unit dosage form that is a capsule. In some embodiments, the capsule comprises a capsule shell comprising a polymeric shell. In some embodiments, the polymeric shell comprises hypromellose and titanium dioxide.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5%” indicates a range of from 4.5 to 5.5 %.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HBRM-associated factors complex in a human cell.

As used herein, the term “BAF complex-related disorder” refers to a disorder that is caused or affected by the level of activity of a BAF complex.

As used herein, the term “BRG1 activity” refers to the BRG1 enzyme ATPase activity.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

The term “CTLA-4 inhibitor,” as used herein, refers to a compound such as an antibody capable of inhibiting the activity of the protein that in humans is encoded by the CTLA4 gene. Known CTLA-4 inhibitors include ipilimumab.

The term “CYP3A inhibitor,” as used herein, refers to a compound capable of inhibiting the activity of the protein that in humans is encoded by the human gene locus of cytochrome P450, family 3, subfamily A. Representative examples of CYP3A proteins include CYP3A4 and CYP3A5. Strong CYP3A inhibitors are those CYP3A inhibitors that increase the AUC of a sensitive CYP3A substrate≥5-fold.

The term “CYP3A inducer,” as used herein, refers to a compound capable of inducing the activity of the protein that in humans is encoded by the human gene locus of cytochrome P450, family 3, subfamily A. Representative examples of CYP3A proteins include CYP3A4 and CYP3A5. Strong CYP3A inducers are those CYP3A inducers that decrease the AUC of a sensitive CYP3A substrate≥80%.

By a “decreased level” or an “increased level” of a protein or RNA is meant a decrease or increase, respectively, in a protein or RNA level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, pg/mL, ng/mL) or percentage relative to total protein in a sample.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

A cancer “determined to be drug resistant,” as used herein, refers to a cancer that is drug resistant, based on unresponsiveness or decreased responsiveness to a chemotherapeutic agent, or is predicted to be drug resistant based on a prognostic assay (e.g., a gene expression assay).

By a “drug resistant” is meant a cancer that does not respond, or exhibits a decreased response to, one or more chemotherapeutic agents (e.g., any agent described herein).

As used herein, the term “failed to respond to a prior therapy” or “refractory to a prior therapy,” refers to a cancer that progressed despite treatment with the therapy.

As used herein, the term “inhibiting BRM” and/or “inhibiting BRG1” refers to blocking or reducing the level or activity of the ATPase catalytic binding domain or the bromodomain of the protein. BRM and/or BRG1 inhibition may be determined using methods known in the art, e.g., a BRM and/or BRG1 ATPase assay, a Nano DSF assay, or a BRM and/or BRG1 Luciferase cell assay.

As used herein, the term “LXS196,” also known as IDE196, refers to the PKC inhibitor having the structure:

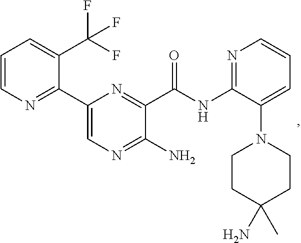 [SMILES: CC1(N)CCN(c2cccnc2NC(=O)c2nc(-c3ncccc3C(F)(F)F)cnc2N)CC1]

or a pharmaceutically acceptable salt thereof.

The term “MEK inhibitor,” as used herein, refers to a compound capable of inhibiting the activity of the mitogen-activated protein kinase enzyme MEK1 or MEK2. An MEK inhibitor may be, e.g., selumetinib, binimetinib, or tametinib.

As used herein, “metastatic nodule” refers to an aggregation of tumor cells in the body at a site other than the site of the original tumor.

As used herein, “metastatic cancer” refers to a tumor or cancer in which the cancer cells forming the tumor have a high potential to or have begun to, metastasize, or spread from one location to another location or locations within a subject, via the lymphatic system or via haematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include but are not limited to lung cancer (e.g., non-small cell lung cancer), breast cancer, ovarian cancer, colorectal cancer, biliary tract cancer, bladder cancer, brain cancer including glioblastomas and medullablastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

The term “narrow therapeutic index,” as used herein, refers to drug compounds, where small differences in dose or blood concentration may lead to serious therapeutic failures or adverse drug reactions that are life-threatening or result in persistent or significant disability or incapacity. A therapeutic index is a numerical value comparing therapeutically effective doses to the toxicity-causing doses. For example, a therapeutic index may be represented as a ratio of a median toxic dose over median effective dose.

“Non-metastatic cell migration cancer” as used herein refers to cancers that do not migrate via the lymphatic system or via haematogenous spread.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

The term “PKC inhibitor,” as used herein, refers to a compound capable of inhibiting the activity of the protein kinase C. A PKC inhibitor may be, e.g., sotrastaurin or IDE196.

“Proliferation” as used in this application involves reproduction or multiplication of similar forms (cells) due to constituting (cellular) elements.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

The term “sensitive substrate” of a protein, as used herein, refers to a compound, whose area under the concentration-time curve (AUC) values increase 5-fold or more when co-administered with a known inhibitor of the protein, or whose AUC ratio in poor metabolizers for the protein is≥5-fold compared to extensive metabolizers.

The term “sensitive CYP3A substrate,” as used herein, refers to a compound, whose area under the concentration-time curve (AUC) values increase 5-fold or more when co-administered with a known CYP3A inhibitor, or whose AUC ratio in poor metabolizers for a CYP3A enzyme is≥5-fold compared to extensive metabolizers.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a graph illustrating inhibition of cell proliferation of several cancer cell lines by a BRG1/BRM inhibitor (Compound A).

FIG. 2A is a graph illustrating inhibition of cell proliferation of uveal melanoma cell line 92-1 by a BRG1/BRM inhibitor (Compound A), a MEK inhibitor (selumetinib), and a PKC inhibitor (LXS196).

FIG. 2B is a graph illustrating inhibition of cell proliferation of uveal melanoma cell line 92-1 by a BRG1/BRM inhibitor (the compound of formula (I)).

FIG. 3 is a graph illustrating inhibition of cell proliferation of uveal melanoma cell line MP41 by a BRG1/BRM inhibitor (Compound A), a MEK inhibitor (selumetinib), and a PKC inhibitor (LXS196).

FIG. 4 is a graph illustrating inhibition of cell proliferation of several cancer cell lines by a BRG1/BRM inhibitor (Compound B).

FIG. 5 is a graph illustrating the area under the curves (AUCs) calculated from dose-response curves for cancer cell lines treated with a BRG1/BRM inhibitor (Compound B).

FIG. 6 is a graph illustrating inhibition of cell proliferation of uveal melanoma and non-small cell lung cancer cell lines by a BRG1/BRM inhibitor (Compound B).

FIG. 7 is a graph illustrating inhibition of cell proliferation of uveal melanoma cell line 92-1 by a BRG1/BRM inhibitor (Compound B), a MEK inhibitor (selumetinib), and a PKC inhibitor (LXS196).

FIG. 8 is a graph illustrating inhibition of cell proliferation of uveal melanoma cell line MP41 by a BRG1/BRM inhibitor (Compound B), a MEK inhibitor (selumetinib), and a PKC inhibitor (LXS196).

FIG. 9 is a graph illustrating inhibition of cell proliferation of parental and PKC-inhibitor refractory uveal melanoma cell lines by a PKC inhibitor (LXS196).

FIG. 10 is a graph illustrating inhibition of cell proliferation of parental and PKC-inhibitor refractory uveal melanoma cell lines by a BRG1/BRM inhibitor (Compound B).

FIG. 11 is a graph illustrating inhibition of tumor growth in mice engrafted with uveal melanoma cell lines by a BRG1/BRM inhibitor (Compound C).

FIG. 12 is an illustration of the size of tumors from mice engrafted with uveal melanoma cell lines and dosed with a BRG1/BRM inhibitor (Compound C).

FIG. 13 is a graph illustrating body weight change of mice engrafted with uveal melanoma cell lines and dosed with a BRG1/BRM inhibitor (Compound C).

FIG. 14 is a graph illustrating inhibition of cell proliferation of several uveal melanoma cell lines by N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1H-pyrrole-3-carboxamide.

FIG. 15 is a graph illustrating inhibition of tumor growth in mice engrafted with uveal melanoma cell lines by N-((S)-14(4-(6-(cis-2,6-dimethylmorpholino)pyridin-2-yl)thiazol-2-yl)amino)-3-methoxy-1-oxopropan-2-yl)-1-(methylsulfonyl)-1H-pyrrole-3- carboxamide.

FIG. 16 is a graph illustrating body weight change of mice engrafted with uveal melanoma cell lines and dosed with N-((S)-14(4-(6-(cis-2,6-dimethylmorpholino)pyridin-2-yl)thiazol-2-yl)amino)-3-methoxy-1-oxopropan-2-yl)-1-(methylsulfonyl)-1H-pyrrole-3- carboxamide.

### DETAILED DESCRIPTION

In general, the invention provides methods of administering the compound of formula (I) in a therapeutic regimen to a subject in need thereof, e.g., for treating cancer (e.g., uveal melanoma, metastatic uveal melanoma):

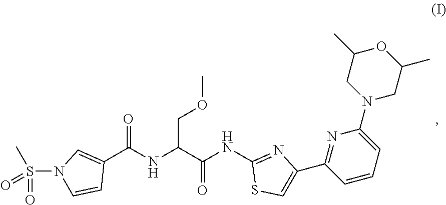 [SMILES: COCC(NC(=O)c1ccn(S(C)(=O)=O)c1)C(=O)Nc1nc(-c2cccc(N3CC(C)OC(C)C3)n2)cs1]

where the compounds of formula (I) is administered to the subject in a therapeutic regimen including a therapeutically effective amount of the compound of formula (I), or a pharmaceutically acceptable salt thereof, without concomitant administration of a CYP3A inhibitor (e.g., a strong CYP3A inhibitor), a CYP3A inducer (e.g., a strong CYP3A inducer), a sensitive CYP3A substrate with a narrow therapeutic index, a sensitive P-gp substrate with a narrow therapeutic index, a sensitive BCRP substrate with a narrow therapeutic index, or a combination thereof.

Advantageously, the methods of the invention provide a therapeutically effective amount of the compound of formula (I) to a subject in need thereof without compromising its efficacy or safety by eliminating concomitant administration of drug classes (e.g., CYP3A inhibitors (e.g., strong CYP3A inhibitors) and CYP3A inducers (e.g., strong CYP3A inducers)) capable of adversely affecting pharmacokinetics or pharmacodynamics of the compound of formula (I). Furthermore, the methods of the invention may eliminate possible adverse effects of the compound of formula (I) upon compounds (e.g., sensitive CYP3A substrates with a narrow therapeutic index, sensitive P-gp substrates with a narrow therapeutic index, and sensitive BCRP substrates with a narrow therapeutic index) that may be pharmacokinetically sensitive to the compound of formula (I).

A strong CYP3A inhibitor may be, e.g., boceprevir, cobicistat, danoprevir, elvitegravir, grapefruit juice, indinavir, itraconazole, ketoconazole, lopinavir, paritaprevir, ombitasvir, dasabuvir, posaconazole, ritonavir, saquinavir, telaprevir, tipranavir, telithromycin, troleandomycin, voriconazole, clarithromycin, idelalisib, nefazodone, nelfinavir, or a pharmaceutically acceptable salt thereof, or a combination thereof. A strong CYP3A inducer may be, e.g., apalutamide, carbamazepine, enzalutamide, mitotane, phenytoin, rifampin, St. John's wort, or a pharmaceutically acceptable salt thereof, or a combination thereof. A sensitive CYP3A substrate with a narrow therapeutic index may be, e.g., alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, tolvaptan, or a pharmaceutically acceptable salt thereof, or a combination thereof. A sensitive P-gp substrate with a narrow therapeutic index may be, e.g., dabigatran etexilate, digoxin, fexofenadine, loperamide, quinidine, talinolol, vinblastine, or a pharmaceutically acceptable salt thereof, or a combination thereof. A BCRP substrate with a narrow therapeutic index may be, e.g., coumestrol, daidzein, dantrolene, estrone-3-sulfate, genistein, prazosin, sulfasalazine, rosuvastatin, or a pharmaceutically acceptable salt thereof, or a combination thereof.

Additionally, in the methods of the invention, the therapeutic regimen may be administered without concomitant administration of an acid-reducing agent (e.g., an antacid, H2 blocker, proton pump inhibitor, or a combination thereof), provided that, an acid-reducing agent that is an antacid may be concomitantly administered with the therapeutic regimen in a staggered dosing manner. For the antacid, the staggered dosing manner typically entails separation of the administrations of the compound of formula (I) from the antacid administrations by at least 2 hours.

### Methods

The compound of formula (I) is useful in the methods of the invention and, while not bound by theory, is believed to exert its ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the present invention relates to methods of treating melanoma (e.g., uveal melanoma), prostate cancer, breast cancer, bone cancer, renal cell carcinoma, or a hematologic cancer.

In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×x).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating fora population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, hematologic cancer, and penile cancer.

### COMBINATION FORMULATIONS and USES THEREOF

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, ad renocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma!l and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MED14736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK1, CHK2, A2aR, B-7 family ligands, or a combination thereof.

In some embodiments, the compound of the invention is used in combination with another anti-cancer therapy used for the treatment of uveal melanoma such as surgery, a MEK inhibitor, and/or a PKC inhibitor, or a combination thereof. For example, in some embodiments, the method further comprises performing surgery prior to, subsequent to, or at the same time as administration of the compound of the invention. In some embodiments, the method further comprises administration of a MEK inhibitor (e.g., selumetinib, binimetinib, or tametinib) and/or a PKC inhibitor (e.g., sotrastaurin or IDE196) prior to, subsequent to, or at the same time as administration of the compound of the invention. In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

A compound described herein may be formulated into pharmaceutical compositions for administration to human subjects in a biologically compatible form suitable for administration in vivo. Pharmaceutical compositions typically include an active agent as described herein and a physiologically acceptable excipient (e.g., a pharmaceutically acceptable excipient). Formulation principles for the compound of formula (I) have been described in WO 2020/160180, the disclosure of which is incorporated by reference herein in its entirety.

The compound of formula (I) of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. Preferably, the compound of formula (I) is administered orally.

Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary).

The pharmaceutical composition may contain suitable pharmaceutical carriers and excipients as described herein. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. The unit dosage form may contain one or more of a filler, a disintegrant, a wetting agent, a glidant, a lubricant, and a capsule shell.

In some examples, the compound of formula (I) or a pharmaceutically acceptable salt thereof may be 2.5 to 20% (w/w) of the pharmaceutical composition.

In some examples, the filler may be 70 to 90% (w/w) of the pharmaceutical composition. The filler may be microcrystalline cellulose, mannitol, or a combination thereof.

In some examples, the disintegrant may be 4 to 6% (w/w) of the pharmaceutical composition. The disintegrant may be croscarmellose sodium.

In some examples, the wetting agent may be 0.5 to 1.5% (w/w) of the pharmaceutical composition. The wetting agent may be sodium lauryl sulfate.

In some examples, the glidant may be 1.5 to 2.5% (w/w) of the pharmaceutical composition. The glidant may be colloidal silicon dioxide.

In some examples, the lubricant may be 0.4 to 0.6% (w/w) of the pharmaceutical composition. The lubricant may be magnesium stearate.

In some examples, the capsule shell is made of a polymeric shell. The polymeric shell can be made from hypromellose and titanium dioxide.

In some examples, the pharmaceutical composition comprises:
2.5 to 20% (w/w) of the compound of formula (I) or a pharmaceutically acceptable salt thereof;
70 to 90% (w/w) of the filler;
4 to 6% (w/w) of the disintegrant;
0.5 to 1.5% (w/w) of the wetting agent;
1.5 to 2.5% (w/w) of the glidant; and
0.4 to 0.6% (w/w) of the lubricant.

In some examples, the pharmaceutical composition comprises:
2.5 to 20% (w/w) of the compound of formula (I) ora pharmaceutically acceptable salt thereof;
70 to 90% (w/w) of microcrystalline cellulose, mannitol, or a combination thereof;
4 to 6% (w/w) of croscarmellose sodium;
0.5 to 1.5% (w/w) of sodium lauryl sulfate;
1.5 to 2.5% (w/w) of colloidal silicon dioxide; and
0.4 to 0.6% (w/w) of magnesium stearate.

In some examples, the pharmaceutical composition comprises 2.5 mg to 20 mg of the compound of formula (I) or a pharmaceutically acceptable salt thereof.

In some examples, the pharmaceutical composition comprises:
2.6% (w/w) of the compound of formula (I) or a pharmaceutically acceptable salt thereof;
50.8% (w/w) of microcrystalline cellulose;
38.1% (w/w) of mannitol;
5% (w/w) of croscarmellose sodium;
1.0% (w/w) of sodium lauryl sulfate;
2.0% (w/w) of colloidal silicon dioxide; and
0.5% (w/w) of magnesium stearate.

In some examples, the pharmaceutical composition comprises:
20% (w/w) of the compound of formula (I) or a pharmaceutically acceptable salt thereof;
48.2% (w/w) of microcrystalline cellulose;
37.0% (w/w) of mannitol;
5.8% (w/w) of croscarmellose sodium;
1.2% (w/w) of sodium lauryl sulfate;
2.3% (w/w) of colloidal silicon dioxide; and
0.6% (w/w) of magnesium stearate.

In some examples, the pharmaceutical composition comprises 2.5 mg of the compound of formula (I) or a pharmaceutically acceptable salt thereof. In some examples, the pharmaceutical composition comprises 20 mg of the compound of formula (I) or a pharmaceutically acceptable salt thereof. In some examples, the pharmaceutical composition is a unit dosage form that is a capsule. In some examples, the capsule comprises a capsule shell comprising a polymeric shell. In some examples, the polymeric shell comprises hypromellose and titanium dioxide.

### Unit Dosage Forms

A compound described herein may be formulated into a unit dosage form for oral administration (e.g., a capsule). The compound of formula (I) may be supplied in different capsule strengths for oral administration (e.g., 1 to 2.5 mg, 2.5 to 5 mg, 5 to 10 mg, 10 to 15 mg, 15 to 20 mg, 20 to 25 mg, or 50 to 100 mg). In some examples, the compound of formula (I) is supplied in 2.5 mg or 20 mg capsule strengths for oral administration.

The unit dosage form may contain suitable pharmaceutical carriers and excipients as described in the pharmaceutical compositions section. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. The unit dosage form may contain one or more of a filler, a disintegrant, a wetting agent, a glidant, a lubricant, and a capsule shell.

In some examples, the compound of formula (I) or a pharmaceutically acceptable salt thereof may be 2.5 to 20% (w/w) of the unit dosage form.

In some examples, the filler may be 70 to 90% (w/w) of the unit dosage form. The filler may be microcrystalline cellulose, mannitol, or a combination thereof.

In some examples, the disintegrant may be 4 to 6% (w/w) of the unit dosage form. The disintegrant may be croscarmellose sodium.

In some examples, the wetting agent may be 0.5 to 1.5% (w/w) of the unit dosage form. The wetting agent may be sodium lauryl sulfate.

In some examples, the glidant may be 1.5 to 2.5% (w/w) of the unit dosage form. The glidant may be colloidal silicon dioxide.

In some examples, the lubricant may be 0.4 to 0.6% (w/w) of the unit dosage form. The lubricant may be magnesium stearate.

In some examples, the capsule shell is made of a polymeric shell. The polymeric shell can be made from hypromellose and titanium dioxide.

In some examples, the unit dosage form comprises:
2.5 to 20% (w/w) of the compound of formula (I) or a pharmaceutically acceptable salt thereof;
70 to 90% (w/w) of the filler;
4 to 6% (w/w) of the disintegrant;
0.5 to 1.5% (w/w) of the wetting agent;
1.5 to 2.5% (w/w) of the glidant; and
0.4 to 0.6% (w/w) of the lubricant.

In some examples, the unit dosage form comprises:
2.5 to 20% (w/w) of the compound of formula (I) or a pharmaceutically acceptable salt thereof;
70 to 90% (w/w) of microcrystalline cellulose, mannitol, or a combination thereof;
4 to 6% (w/w) of croscarmellose sodium;
0.5 to 1.5% (w/w) of sodium lauryl sulfate;
1.5 to 2.5% (w/w) of colloidal silicon dioxide; and
0.4 to 0.6% (w/w) of magnesium stearate.

In some examples, the unit dosage form comprises 2.5 mg to 20 mg of the compound of formula (I) or a pharmaceutically acceptable salt thereof.

In some examples, the unit dosage form comprises:
2.6% (w/w) of the compound of formula (I) or a pharmaceutically acceptable salt thereof;
50.8% (w/w) of microcrystalline cellulose;
38.1% (w/w) of mannitol;
5% (w/w) of croscarmellose sodium;
1.0% (w/w) of sodium lauryl sulfate;
2.0% (w/w) of colloidal silicon dioxide; and
0.5% (w/w) of magnesium stearate.

In some examples, the pharmaceutical composition comprises:
20% (w/w) of the compound of formula (I) or a pharmaceutically acceptable salt thereof;
48.2% (w/w) of microcrystalline cellulose;
37.0% (w/w) of mannitol;
5.8% (w/w) of croscarmellose sodium;
1.2% (w/w) of sodium lauryl sulfate;
2.3% (w/w) of colloidal silicon dioxide; and
0.6% (w/w) of magnesium stearate.

In some examples, the unit dosage form comprises 2.5 mg of the compound of formula (I) or a pharmaceutically acceptable salt thereof. In some examples, the unit dosage form comprises 20 mg of the compound of formula (I) or a pharmaceutically acceptable salt thereof. In some examples, the unit dosage form that is a capsule. In some examples, the capsule comprises a capsule shell comprising a polymeric shell. In some examples, the polymeric shell comprises hypromellose and titanium dioxide.

The compound described herein may be formulated into a unit dosage form for oral administration (e.g., a capsule) as described in Table 1.

TABLE 1

| | | 2.5 mg Capsule | | 20 mg Capsule | |
|---|---|---|---|---|---|
| | | Amount per | | Amount per | |
| | | Capsule | Content | Capsule | Content |
| Component | Function | (mg) | (%) | (mg) | (%) |
| Compound of formula (I) | Active | 2.50 | 2.63 | 20.00 | 17.39 |
| | ingredient | | | | |
| Microcrystalline cellulose | Filler | 48.24 | 50.78 | 48.20 | 41.91 |
| Mannitol | Filler | 36.18 | 38.08 | 36.95 | 32.13 |
| Croscarmellose Sodium | Disintegrant | 4.75 | 5.00 | 5.80 | 5.04 |
| Sodium lauryl sulfate | Wetting | 0.95 | 1.00 | 1.15 | 1.00 |
| | agent | | | | |
| Colloidal silicon dioxide | Glidant | 1.90 | 2.00 | 2.30 | 2.00 |
| Magnesium stearate | Lubricant | 0.48 | 0.51 | 0.60 | 0.52 |
| Size 4, Swedish Orange | Capsule | 1 capsule | — | — | — |
| HPMC Capsules | Shell | | | | |
| Size 3, Blue Green | Capsule | — | — | 1 capsule | — |
| HPMC Capsules | Shell | | | | |
| Total | | 95.00 | 100 | 115.00 | 100 |

The composition of the Swedish orange hypromellose capsule shells is described in Table 2.

TABLE 2

| Composition of | | |
|---|---|---|
| capsule body and cap | Function | Content |
| FDA/E172 RED | Colorant | 1.1817% |
| IRON OXIDE | | |
| TITANIUM DIOXIDE | Opacifier | 0.4916% |
| Hypromellose | Structure | qs 100% |

The composition of blue green hypromellose capsule shells is described in Table 3.

TABLE 3

| Composition of | | |
|---|---|---|
| capsule body and cap | Function | Content |
| FD&C Blue #1 | Colorant | 0.0281% |
| FD&C Yellow #5 | Colorant | 0.0069% |
| TITANIUM DIOXIDE | Opacifier | 2.2306% |
| Hypromellose | Structure | qs 100% |

### EXAMPLES

The abbreviations below are used throughout the examples section.

| | |
|---|---|
| Boc | tert-butoxycarbonyl |
| DCM | dichloromethane |
| DIPEA or DIEA | N.N-diisopropylethylamine |
| DMF | N.N-dimethylformamide |
| DMSO | dimethyl sulfoxide |
| EDCI | N-(3-dimethylaminopropyl)-N′-ethylcarbodiimide |
| | hydrochloride |
| EEDQ | 2-ethoxy-1-ethoxycarbonyl-1,2-dihydroquinoline |
| EtOH | ethyl alcohol |
| h or hr | hour |
| HOBt or HOBT | 1-hydroxybenzotriazole hydrate |
| MeOH | methyl alcohol |
| MsCl | methanesulfonyl chloride |
| NaHMDS | sodium bis(trimethylsilyl)amide |
| PdCl₂(dtbpf) | dichloro[1,1′-bis(di-t- |
| | butylphosphino)ferrocene]palladium(II) |
| THF | tetrahydrofuran |
| TMSCHN₂ | (diazomethyl)trimethylsilane |

### Example 1. Preparation of N-((S)-14(4-(6-(cis-2,6-dimethylmorpholino)pyridin-2-yl)thiazol-2-yl)amino)-3-methoxy-1-oxopropan-2-yl)-1-(methylsulfonyl)-1H- pyrrole-3-carboxamide

N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1H-pyrrole-3-carboxamide was synthesized as shown in Scheme 1 below.

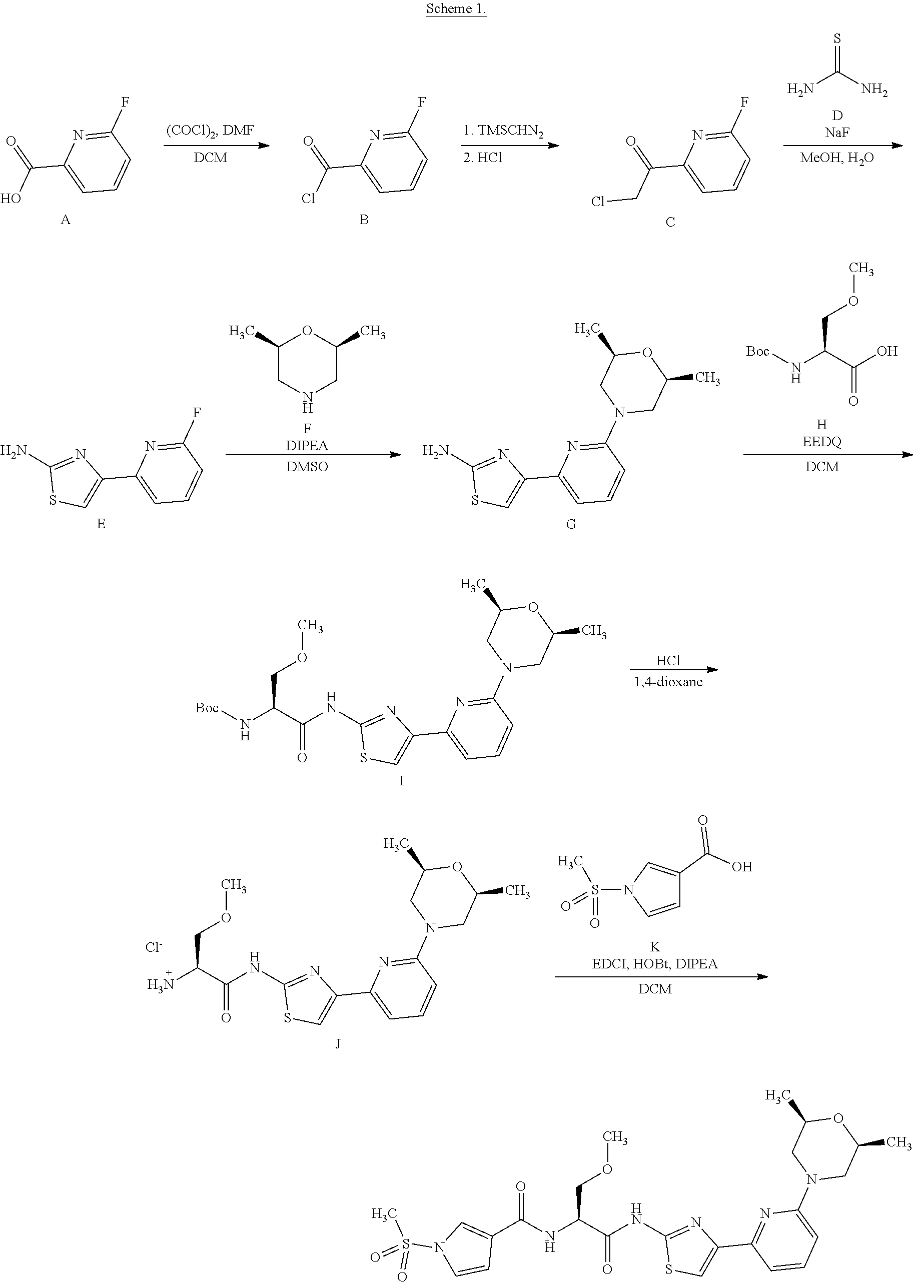

### Step 1: Preparation of 6-fluoropyridine-2-carbonyl Chloride (Intermediate B)

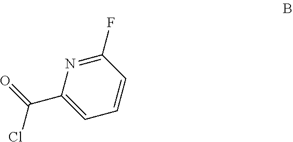

To a cooled (0° C.) solution of 6-fluoropyridine-2-carboxylic acid (50.0 g, 354 mmol) in dichloromethane (500 mL) and N,N-dimethylformamide (0.26 mL, 3.54 mmol) was added oxalyl chloride (155 mL, 1.77 mol). After complete addition of oxalyl chloride, the reaction mixture was warmed to room temperature. After 0.5 hours, the mixture was concentrated under vacuum to give Intermediate B (56.50 g) as a white solid, which was used in the next step without further purification.

### Step 2: Preparation of 2-chloro-1-(6-fluoro-2-pyridyl)Ethenone (Intermediate C)

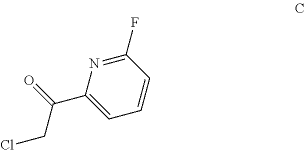 [SMILES: O=C(CCl)c1cccc(F)n1]

To a cooled (0° C.) mixture of Intermediate B (56.0 g, 351 mmol) in 1,4-dioxane (800 mL) was added in a dropwise manner a solution of 2M trimethylsilyl diazomethane in hexanes (351 mL, 702 mmol). The resulting reaction mixture was stirred at 25° C. for 10 h. The reaction mixture was subsequently quenched with a solution of 4M HCl in 1,4-dioxane (500 mL, 2.0 mol). After stirring for 2 h, the reaction solution was concentrated under vacuum to give an oil. The residue was diluted with saturated aqueous NaHCO₃ and extracted three times with ethyl acetate. The combined organic layers were washed twice with brine, dried over Na₂SO₄, filtered, and concentrated under reduced pressure to give Intermediate C (35.5 g) as a white solid, which was used to next step directly.

### Step 3: Preparation of 4-(6-fluoro-2-pyridyl)thiazol-2-amine (Intermediate E)

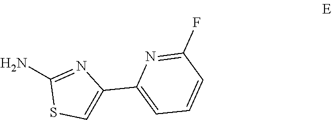 [SMILES: Nc1nc(-c2cccc(F)n2)cs1]

To a solution of Intermediate C (35.5 g, 205 mmol) and thiourea (14.0 g, 184 mmol) in a mixture of methanol (250 mL) and water (250 mL) at room temperature was added NaF (3.56 g, 84.8 mmol). After stirring for 0.5 h, the reaction mixture was partially concentrated under vacuum to remove MeOH, and the resulting solution was acidified to pH˜3 with aqueous 2M HCl. After 15 minutes, the solution was extracted three times with ethyl acetate. The organic layers were discarded and the aqueous phase was alkalized with saturated aqueous NaHCO₃ and stirred for 30 minutes, and extracted three times with ethyl acetate. The combined organic layers were washed three times with brine, dried over Na₂SO₄, filtered, and concentrated under reduced pressure. The residue was triturated with petroleum ether and stirred at 25° C. for 10 minutes and filtered. The resultant solids were dried under vacuum to give Intermediate E (28.0 g, 143 mmol, 70.1% yield, 100% purity) as a white solid.
LCMS (ESI) m/z: [M+H]⁺=195.8.
¹H NMR (400 MHz, DMSO-d₆) δ 8.00-7.96 (m, 1H), 7.72 (d, J=7.2 Hz, 1H), 7.24 (s, 1H), 7.16 (s, 2H), 7.02 (d, J=8.0 Hz, 1H).

### Step 4: Preparation of 4-[6-[cis-2,6-dimethylmorpholin-4-yl]-2-pyridyl]thiazol-2-amine (Intermediate G)

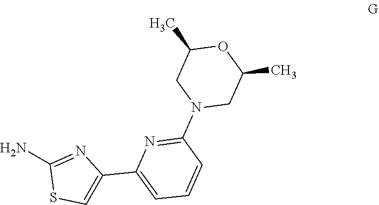 [SMILES: C[C@@H]1CN(c2cccc(-c3csc(N)n3)n2)C[C@H](C)O1]

Ten separate mixtures of Intermediate E (2.00 g, 10.3 mmol), cis-2,6-dimethylmorpholine (3.54 g, 30.7 mmol), and DIPEA (5.35 mL, 30.7 mmol) in dimethyl sulfoxide (10 mL) were stirred in parallel at 120° C. under N₂ atmosphere. After 36 h, the reaction mixtures were combined and added dropwise to water. The resulting suspension was filtered and the filter cake was washed three times with water and once with petroleum ether, then dried over under reduced pressure to give Intermediate G (25.5 g, 87.8 mmol, 95.2% yield) as a yellow solid.
LCMS (ESI) m/z: [M+H]⁺=291.2.
¹H NMR (400 MHz, DMSO-d₆) δ 7.56-7.54 (m, 1H), 7.17 (s, 1H), 7.13 (d, J=7.6 Hz, 1H), 7.01 (s, 2H), 6.72 (d, J=8.8 Hz, 1H), 4.26-4.15 (m, 2H), 3.67-3.55 (m, 2H), 2.38-2.34 (m, 2H), 1.17 (d, J=6.4 Hz, 6H).

### Step 5: Preparation of tert-butyl N-[(1S)-2-[[4-[6-[cis-2,6-dimethylmorpholin-4-yl]-2-pyridyl]thiazol-2-yl]amino]-1-(methoxymethyl)-2-oxo- ethyl]carbamate (Intermediate I)

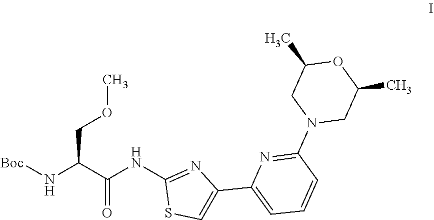 [SMILES: COC[C@H](NC(=O)OC(C)(C)C)C(=O)Nc1nc(-c2cccc(N3C[C@@H](C)O[C@@H](C)C3)n2)cs1]

To a solution of Intermediate G (12.0 g, 41.3 mmol) and (2S)-2-(tertbutoxycarbonylamino)-3-methoxy-propanoic acid (10.9 g, 49.6 mmol) in dichloromethane (60 mL) was added EEDQ (12.3 g, 49.6 mmol). After stirring at room temperature for 16 h, the reaction mixture was concentrated under reduced pressure to give a residue. The residue was purified by silica gel column chromatography (petroleum ether:ethyl acetate=2:1 to 3:2) to give Intermediate I (20.0 g, 40.7 mmol, 98.5% yield) as a yellow gum.
LCMS (ESI) m/z: [M+H]⁺=492.2.
¹H NMR (400 MHz, DMSO-d₆) δ 12.37 (s, 1H), 7.78 (s, 1H), 7.64-7.60 (m, 1H), 7.25 (d, J=7.2 Hz, 1H),
7.16 (d, J=7.2 Hz, 1H), 6.79 (d, J=8.4 Hz, 1H), 4.50-4.48 (m, 1H), 4.25 (d, J=11.6 Hz, 2H), 3.70-3.51 (m, 4H), 3.26 (s, 3H), 2.44-2.40 (m, 2H), 1.39 (s, 9H), 1.18 (d, J=6.4 Hz, 6H).

### Step 6: Preparation of (S)-4-(4-(6-(cis-2,6-dimethylmorpholino)pyridin-2-yl)thiazol-2-yl)-1-methoxy-3-oxobutan-2-aminium chloride (Intermediate J)

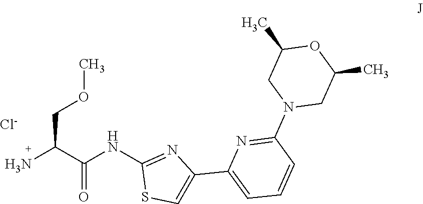 [SMILES: [Cl-] ; COC[C@H](N)C(=O)Nc1nc(-c2cccc(N3C[C@@H](C)O[C@@H](C)C3)n2)cs1]

To a solution of 4M HCl in 1,4-dioxane (200 mL, 800 mmol) was added a solution of Intermediate I (20.0 g, 40.7 mmol) in dichloromethane (50 mL). After stirring at room temperature for 2 h, the mixture was diluted with methyl tert-butyl ether resulting in a suspension. The solid was collected by filtration, washed twice with methyl tert-butyl ether, and dried in vacuo to give Intermediate J (19.0 g) as a yellow solid, which was used in the next step without further purification.
LCMS (ESI) m/z: [M+H]⁺=392.3.
¹H NMR (400 MHz, DMSO-d₆) δ 13.44-12.30 (m, 1H), 8.65 (d, J=4.4 Hz, 3H), 7.87 (s, 1H), 7.66-7.64
(m, 1H), 7.25 (d, J=7.2 Hz, 1H), 6.83 (d, J=8.8 Hz, 1H), 4.39-4.30 (m, 1H), 4.25 (d, J=11.6 Hz, 2H), 3.94-3.86 (m, 1H), 3.85-3.77 (m, 1H), 3.69-3.57 (m, 2H), 3.31 (s, 3H), 2.43 (m, 2H), 1.18 (d, J=6.4 Hz, 6H).

### Preparation of 1-(methylsulfonyl)-1H-pyrrole-3-carboxylic acid (Intermediate K)

1-(methylsulfonyl)-1H-pyrrole-3-carboxylic acid was synthesized as shown in Scheme 2 below.

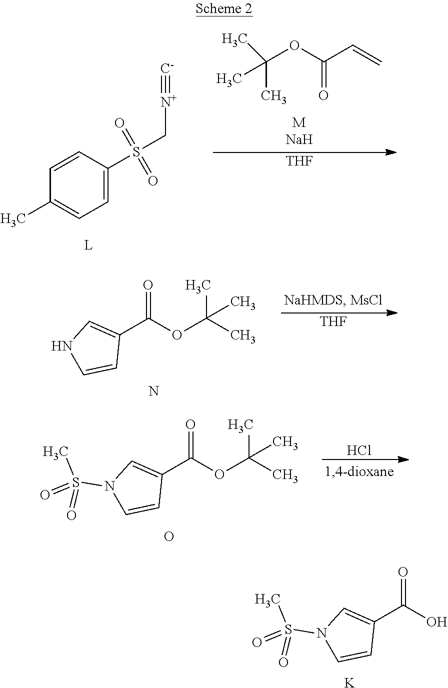

### Step A: Preparation of tert-butyl 1H-pyrrole-3-carboxylate (Intermediate N)

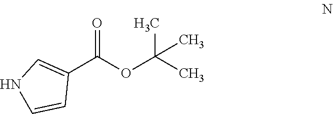

To a mixture of tert-butyl-prop-2-enoate (78.6 mL, 542 mmol) and 1-(isocyanomethylsulfonyl)-4-methylbenzene (106 g, 542 mmol) in THF (1300 mL) was added 60% NaH in mineral oil (25.97 g, 649 mmol) slowly at 30° C. over 1 hour and then heated to 70° C. After 2 h, the reaction mixture was poured into saturated aqueous NH₄Cl solution and extracted three times with ethyl acetate. The combined organic phase was washed twice with brine, dried over anhydrous Na₂SO₄, filtered, and concentrated under reduced pressure to afford a residue. The residue was purified by silica gel column chromatography (petroleum ether:ethyl acetate=20:1 to 3:1) to afford Intermediate N (41.5 g, 236 mmol, 43% yield) as a yellow solid.
LCMS (ESI) m/z [M+Na]⁺=180.4.
¹H NMR (400 MHz, CDCl₃) δ 8.36 (br s, 1H), 7.35-7.25 (m, 1H), 6.71-6.62 (m, 1H), 6.59-6.49 (m, 1H), 1.48 (s, 9H).

### Step B: Preparation of tert-butyl 1-methylsulfonylpyrrole-3-carboxylate (Intermediate O)

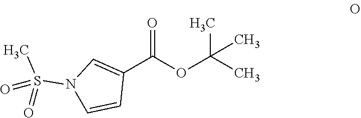 [SMILES: CC(C)(C)OC(=O)c1ccn(S(C)(=O)=O)c1]

To a cooled solution (0° C.) of Intermediate N (40.5 g, 242 mmol) in THF (1500 mL) was added a 1M solution of NaHMDS (484 mL, 484 mmol). After stirring at 0° C. for 30 min, methanesulfonyl chloride (28.1 mL, 363 mmol) was slowly added and the mixture was warmed to 30° C. After 16 h, the reaction mixture was slowly poured into saturated aqueous NH₄Cl solution and extracted three times with ethyl acetate. The combined organic layers were washed twice with brine, dried over anhydrous Na₂SO₄, filtered, and concentrated under reduced pressure to afford a residue. The residue was purified by silica gel chromatography (petroleum ether:ethyl acetate=10:1) to afford a yellow solid. The yellow solid was triturated with methyl tert-butyl ether at room temperature, stirred for 20 minutes, filtered, and dried in vacuum to afford Intermediate O (25.7 g, 105 mmol, 43% yield) as a white solid. ¹H NMR (400 MHz, CDCl₃) δ 7.66-7.64 (m, 1H), 7.10-7.08 (m, 1H), 6.73-6.71 (m, 1H), 3.21 (s, 3H), 1.56 (s, 9H).

### Step C: Preparation of 1-methylsulfonylpyrrole-3-carboxylic acid (Intermediate K)

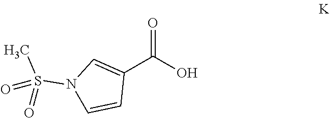

To a mixture of Intermediate O (25.7 g, 105 mmol) in 1,4-dioxane (100 mL) was added a 4M solution of HCl in 1,4-dioxane (400 mL, 1.6 mol) at 15° C. After stirring at at 15° C. for 14 h, the reaction mixture was concentrated under reduced pressure to afford a residue. The residue was triturated with methyl tert-butyl ether at 15° C. for 16 h. The mixture was filtered and dried in vacuum to afford Intermediate K (18.7 g, 98.8 mmol, 94% yield) as a white solid.
LCMS (ESI) m/z [M+H]⁺=189.8.
¹H NMR (400 MHz, methanol-d₄) δ 7.78-7.77 (m, 1H), 7.25-7.23 (m, 1H), 6.72-6.70 (m, 1H), 3.37 (s, 3H).

### Step 7: Preparation of N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1H- pyrrole-3-carboxamide

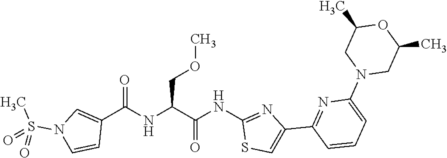 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

To a solution of 1-methylsulfonylpyrrole-3-carboxylic acid (Intermediate K) (2.43 g, 12.9 mmol), EDCI (2.69 g, 14.0 mmol), HOBt (1.89 g, 14.0 mmol), and DIPEA (10.2 mL, 58.4 mmol) in dichloromethane (50 mL) was added Intermediate J (5.00 g, 11.7 mmol). After stirring at room temperature for 4 h, the reaction mixture was concentrated under reduced pressure. The residue was diluted with water and extracted three times with ethyl acetate. The combined organic layers were washed three times with saturated aqueous NH₄Cl, once with brine, dried over Na₂SO₄, filtered, and concentrated under reduced pressure to give a residue. The residue was purified by silica gel column chromatography (petroleum ether:ethyl acetate=1:1 to 1:2). The residue was triturated with methyl tert-butyl ether. After 0.5 h, the suspension was filtered, the filter cake was washed with methyl tert-butyl ether, and dried in vacuo. The solid was dissolved in dimethyl sulfoxide (12 mL) and added dropwise to water (800 mL). The suspension was filtered to give wet filter cake. The filter cake was suspended in water and stirred at room temperature. After 1 hour, the solid was collected by filtration, washed three times with water and dried in vacuo to give N-((S)-1-((4-(6-(cis-2,6-dimethylmorpholino)pyridin-2-yl)thiazol-2-yl)amino)-3-methoxy-1-oxopropan-2-yl)-1-(methylsulfonyl)- 1H-pyrrole-3-carboxamide (3.9 g, 6.93 mmol, 59.3% yield) as a white solid.
LCMS (ESI) m/z: [M+H]⁺=563.1.
¹H NMR (400 MHz, DMSO-d₆) δ 12.49 (br s, 1H), 8.51 (d, J=7.2 Hz, 1H), 7.98-7.97 (m, 1H), 7.78 (s, 1H), 7.67-7.57 (m, 1H), 7.29-7.27 (m, 1H), 7.26 (d, J=7.2 Hz, 1H), 6.88-6.74 (m, 2H), 4.94-4.91 (m, 1H), 4.25 (d, J=11.6 Hz, 2H), 3.77-3.67 (m, 2H), 3.63-3.62 (m, 2H), 3.57 (s, 3H), 3.31 (s, 3H), 2.44-2.38 (m, 2H), 1.18 (d, J=6.0 Hz, 6H).

### Example 2. Potency and Selectivity of ATPase Activity

A series of in vitro experiments were also conducted to determine the potency and selectivity of the compound of formula (I). ATPase activity was assessed in the ADP-Glo assay using a range of concentrations of the compound of formula (I) and a selection of batches. Reaction mixtures were prepared and incubated with the appropriate substrate. After the reactions were stopped, the results were analyzed by measuring luminescence.

These results demonstrated that the compound of formula (I) inhibited BRG1and BRM enzyme ATPase activity with geometric mean half-maximal inhibitory concentration (IC50) values of 6.5 nM (n=11) and 4.5 nM (n=11), respectively. The ATPase domain of chromodomain helicase DNA-binding protein 4 (CHD4) is homologous to that of BRG1 and BRM. The compound of formula (I) did not inhibit the ATPase activity of the CHD4 enzyme at concentrations up to 200 μM (n=3), showing selectivity of the compound of formula (I). Additional profiling indicated that the compound of formula (I) had little binding to 249 other ATPases in cells.

### Example 3. Assay for ATPase catalytic activity of BRM and BRG-1

The ATPase catalytic activity of BRM or BRG-1 was measured by an in vitro biochemical assay using ADP-Glo™ (Promega, V9102). The ADP-Glo™ kinase assay was performed in two steps once the reaction was complete. The first step is to deplete any unconsumed ATP in the reaction. The second step was to convert the reaction product ADP to ATP, which will be utilized by the luciferase to generate luminesce and be detected by a luminescence reader, such as Envision.

The assay reaction mixture (10 μL) contained 30 nM of BRM or BRG-1, 20 nM salmon sperm DNA (from Invitrogen, UltraPure™ Salmon Sperm DNA Solution, cat# 15632011), and 400 μM of ATP in the ATPase assay buffer, which comprises of 20 mM Tris, pH 8, 20 mM MgCl₂, 50 mM NaCl, 0.1% Tween-20, and 1 mM fresh DTT (Pierce™ DTT (Dithiothreitol), cat# 20290). The reaction was initiated by the addition of the 2.5 μL ATPase solution to 2.5 μL ATP/DNA solution on low volume white Proxiplate-384 plus plate (PerkinElmer,cat # 6008280) and incubated at room temperature for 1 hour. Then following addition of 5 μL of ADP-Glo™ Reagent provided in the kit, the reaction incubated at room temperature for 40 minutes. Then 10 μL of Kinase Detection Reagent provided in the kit was added to convert ADP to ATP, and the reaction incubated at room temperature for 60 minutes. Finally, luminescence measurement is collected with a plate-reading luminometer, such as Envision.

BRM and BRG-1 were synthesized from high five insect cell lines with a purity of greater than 90%.

N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1H-pyrrole-3-carboxamide was found to have an 1P₅₀ of 3.9 nM against BRM and 5.2 nM against BRG1 in the assay. N-((R)-1-((4-(6-(cis-2,6-dimethylmorpholino)pyridin-2-yl)thiazol-2-yl)amino)-3-(methoxy-d3)-1-oxopropan-2- yl-3,3-d2)-1-(methylsulfonyl)-1H-pyrrole-3-carboxamide was found to have an IP₅₀ of 443 nM against BRM and 777 nM against BRG1 in the assay. N-((S)-1-((4-(6-(cis-2,6-dimethylmorpholino)pyridin-2-yl)thiazol-2-yl)amino)-3-(methoxy-d₃)-1-oxopropan-2-yl-3,3-d₂)-1-(methylsulfonyl)-1H- pyrrole-3-carboxamide was found to have an IP₅₀ of 4.6 nM against BRM and 7.4 nM against BRG1 in the assay.

### Example 4. Synthesis of Compound A

BRG1/BRM Inhibitor Compound A has the structure:

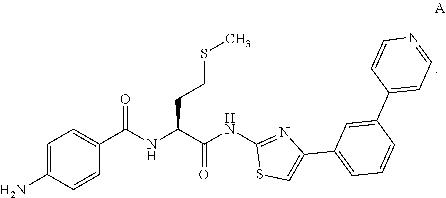 [SMILES: CSCC[C@H](NC(=O)c1ccc(N)cc1)C(=O)Nc1nc(-c2cccc(-c3ccncc3)c2)cs1]

Compound A was synthesized as shown in Scheme 3 below.

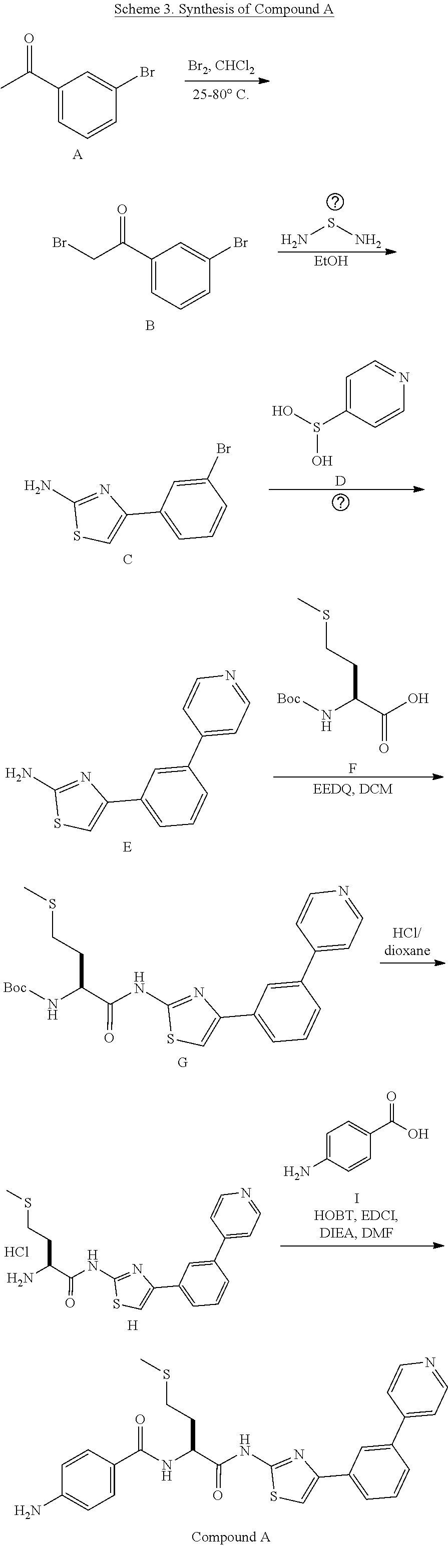

The ATPase catalytic activity of BRM or BRG-1 in the presence of Compound A was measured by the in vitro biochemical assay using ADP-Glo™ (Prometa, V9102) described above. Compound A was found to have an IP₅₀ of 10.4 nM against BRM and 19.3 nM against BRG1 in the assay.

### Example 5. Effects of BRG1/BRM ATPase Inhibition on the Growth of Uveal Melanoma and Hematological Cancer Cell Lines

Procedure: Uveal melanoma cell lines (92-1, MP41, MP38, MP45), prostate cancer cell lines (LNCAP), lung cancer cell lines (NCl-H1299), and immortalized embryonic kidney lines (HEK293T) were plated into 96 well plates with growth media (see Table 4). BRG1/BRM ATPase inhibitor, Compound A, was dissolved in DMSO and added to the cells in a concentration gradient from 0 to 10 μM at the time of plating. Cells were incubated at 37° C. for 3 days. After three days of treatment, the media was removed from the cells, and 30 microliters of TrypLE (Gibco) was added to cells for 10 minutes. Cells were detached from the plates, and resuspended with the addition of 170 microliters of growth media. Cells from two DMSO-treated control wells were counted, and the initial number of cells plated at the start of the experiment, were re-plated into fresh-compound containing plates for an additional four days at 37° C. At day 7, cells were harvested as described above. On day 3 and day 7, relative cell growth was measured by the addition of Cell-titer glo (Prometa), and luminescence was measured on an Envision plate reader (Perkin Elmer). The concentration of compound at which each cell line's growth was inhibited by 50% (Gl₅₀), was calculated using Graphpad Prism, and is plotted below. For multiple myeloma cell lines (OPM2, MM1S, LP1), ALL cell lines (TALL1, JURKAT, RS411), DLBCL cell lines (SUDHL6, SUDHL4, DB, WSUDLCL2, PFEIFFER), AML cell lines (OCIAML5), MDS cell lines (SKM1), ovarian cancer cell lines (OV7, TYKNU), esophageal cancer cell lines (KYSE150), rhabdoid tumor lines (RD, G402. G401, HS729, A204), liver cancer cell lines (HLF, HLE, PLCRPF5), and lung cancer cell lines (SW1573, NCIH2444), the above methods were performed with the following modifications Cells were plated in 96 well plates, and the next day, BRG1/BRM ATPase inhibitor, Compound A, was dissolved in DMSO and added to the cells in a concentration gradient from 0 to 10 μM. At the time of cell splitting on days 3 and 7, cells were split into new 96 well plates, and fresh compound was added four hours after re-plating. Table 4 lists the tested cell lines and growth media used.

TABLE 4
Cell Lines and Growth Media

| Cell Line | Source | Growth Media |
|---|---|---|
| 92-1 | SIGMA | RPMI1640 + 20% FBS |
| A204 | ATCC | McCoy's 5A + 10% FBS |
| DB | ATCC | RPMI1640 + 10% FBS |
| G401 | ATCC | McCoy's 5A + 10% FBS |
| G402 | ATCC | McCoy's 5A + 10% FBS |
| HEK293T | ATCC | DMEM + 10% FBS |
| HLE | JCRB | DMEM + 10% FBS |
| HLF | JCRB | DMEM + 10% FBS |
| HS729 | ATCC | DMEM + 10% FBS |
| JURKAT | ATCC | RPMI1640 + 10% FBS |
| KYSE150 | DSMZ | RPMI1640/Ham's F12 + 10% FBS |
| LNCAP | ATCC | RPMI1640 + 10% FBS |
| LP1 | DSMZ | IMDM + 20% FBS |
| MM1S | ATCC | RPMI1640 + 10% FBS |
| MP38 | ATCC | RPMI1640 + 20% FBS |
| MP41 | ATCC | RPMI1640 + 20% FBS |
| MP46 | ATCC | RPMI1640 + 20% FBS |
| NCIH1299 | ATCC | RPMI1640 + 10% FBS |
| NCIH2444 | ATCC | RPMI1640 + 20% FBS |
| OCIAML5 | DSMZ | alpha-MEM + 20% FBS + 10 ng/ml GM-CSF |
| OPM2 | DSMZ | RPMI1640 + 10% FBS |
| OV7 | ECACC | DMEM/Ham's F12 (1:1) + 2 mM Glutamine + |
| | | 10% FBS + 0.5 ug/ml hydrocortisone + 10 ug/ml |
| | | insulin |
| PFEIFFER | ATCC | RPMI1640 + 10% FBS |
| PLCPRF5 | ATCC | EMEM + 10% FBS |
| RD | ATCC | DMEM + 10% FBS |
| RS411 | ATCC | RPMI1640 + 10% FBS |
| SKM1 | JCRB | RPMI1640 + 10% FBS |
| SUDHL4 | DSMZ | RPMI1640 + 10% FBS |
| SUDHL6 | ATCC | RPMI1640 + 20% FBS |
| SW1573 | ATCC | DMEM + 10% FBS |
| TALL1 | JCRB | RPMI1640 + 10% FBS |
| TYKNU | JCRB | EMEM + 20% FBS |
| WSUDLCL2 | DSMZ | RPMI1640 + 10% FBS |

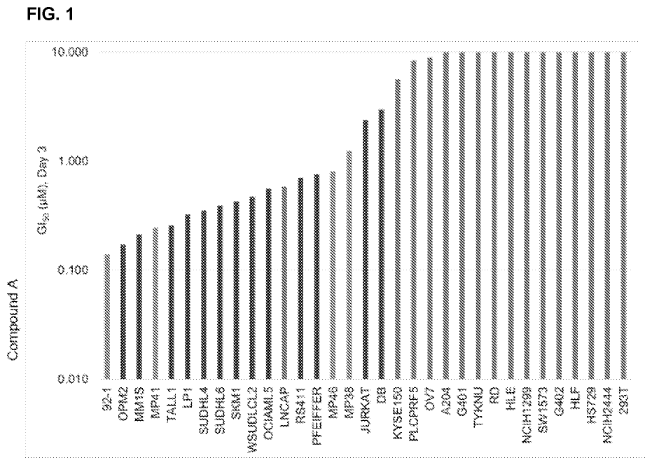

Results: As shown in FIG. 1, the uveal melanoma and hematologic cancer cell lines were more sensitive to BRG1/BRM inhibition than the other tested cell lines. Inhibition of the uveal melanoma and hematologic cancer cell lines was maintained through day 7.

### Example 6. Comparison of BRG1/BRM Inhibitors to Clinical PKC and MEK Inhibitors in Uveal Melanoma Cell Lines

Procedure: Uveal melanoma cell lines, 92-1 or MP41, were plated in 96 well plates in the presence of growth media (see Table 4). BAF ATPase inhibitors (Compound A), PKC inhibitor (LXS196; MedChemExpress), or MEK inhibitor (selumetinib; Selleck Chemicals) were dissolved in DMSO and added to the cells in a concentration gradient from 0 to 10 μM at the time of plating. Cells were incubated at 37° C. for 3 days. After three days of treatment, cell growth was measured with Cell-titer glow (Promega), and luminescence was read on an Envision plate reader (Perkin Elmer).

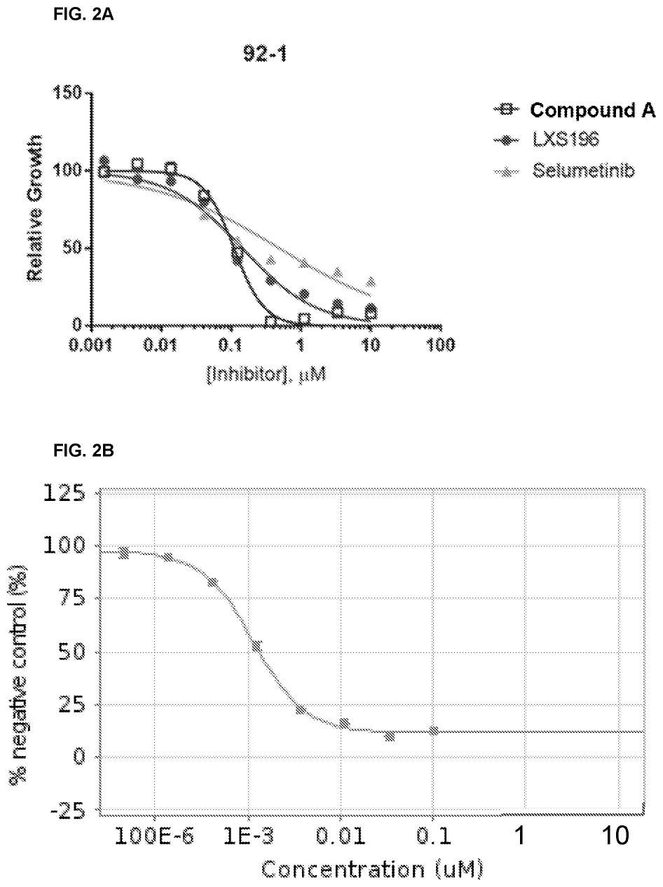

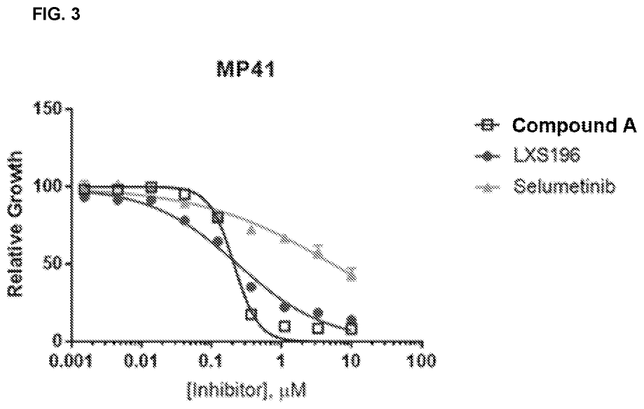

Results: As shown in FIG. 2A and FIG. 3, Compound A showed comparable growth inhibition of uveal melanoma cells as the clinical PKC and MEK inhibitors. Further, Compound A was found to result in a faster onset of inhibition than the clinical PKC and MEK inhibitors.

The Compound of Formula (I) in Uveal Melanoma Cell Line. An in vitro study was performed to investigate the inhibition of cell proliferation in the human UM 92-1 cell line by the compound of formula (I). 92-1 is a cell line derived from a primary human tumor and harbors a mutation in the gene GNAQ, which is a common feature in UM. The compound of formula (I) was dissolved in DMSO and tested at a range of concentrations. The compound of formula (I) was evaluated in a 3-day cell proliferation assay in 92-1 cells, in duplicate, and repeated 11 times. As a negative control, the compound of formula (I) was assayed in triplicate and repeated 5 times in a 10-day proliferation assay in SBC-5 cells that lack the expression of either BRG1 or BRM. At the end of the incubation period for each assay, cell viability was determined using CellTiter-Glo® Luminescent Cell Viability Assay reagent and a plate reader for luminescence detection.

In the three-day cell proliferation assay using 92-1 cells, the mean IC50 of the compound of formula (I) was 1.13 nM (n=11). A representative inhibition curve showing the effect on 92-1 cell proliferation by the compound the compound of formula (I) is shown in FIG. 2B. The mean IC50 of the compound of formula (I) in SBC-5 cells was >25,000 nM. The results demonstrate that the compound of formula (I) potently inhibits the proliferation of a cell line that expresses BRG1 and BRM, while having no significant antiproliferative effect on a cell line which does not have functional BRG1 or BRM. Thus, the antiproliferative effects of the compound of formula (I) are considered due to its inhibitory activity against the BRG1 and BRM proteins.

### Example 7. Synthesis of Compound B

BRG1/BRM Inhibitor Compound B has the structure:

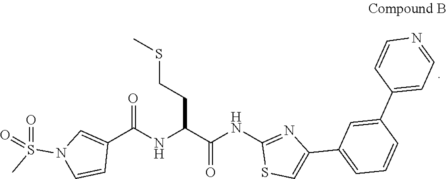

Compound B was synthesized as shown in Scheme 4 below.

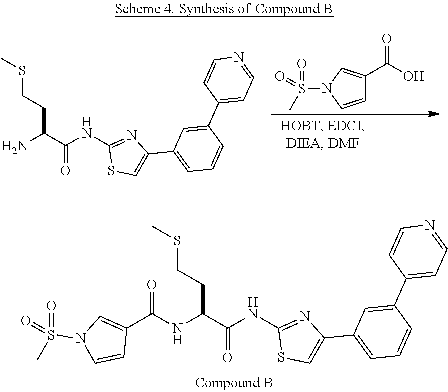

To a mixture of (2S)-2-amino-4-methylsulfanyl-N-[4-[3-(4-pyridyl)phenyl]thiazol-2-yl]butanamide (2 g, 4.75 mmol, HCl salt) and 1-methylsulfonylpyrrole-3-carboxylic acid (898.81 mg, 4.75 mmol) in DMF (20 mL) was added EDCl (1.37 g, 7.13 mmol), HOBt (962.92 mg, 7.13 mmol), and DIEA (2.46 g, 19.00 mmol, 3.31 mL) and the mixture was stirred at 25° C. for 3 h. The mixture was poured into H₂O (100 mL) and the precipitate was collected by filtration. The solid was triturated in MeOH (20 mL) and the precipitate was collected by filtration. The solid was dissolved in DMSO (10 mL) and then the mixture was poured into MeOH (50 mL), and the formed precipitate was collected by filtration and lyophilized to give Compound B (2.05 g, 3.66 mmol, 77.01% yield) as a white solid.
LCMS (ESI) m/z [M+H]⁺=555.9.
¹H NMR (400 MHz, DMSO) δ 12.49 (s, 1H), 8.68-8.66 (m, 2H), 8.46 (d, J=7.2 Hz, 1H), 8.31-8.30 (m, 1H), 8.02-8.00 (m, 1H), 7.94-7.96 (m, 1H), 7.83 (s, 1H), 7.73-7.74 (m, 3H), 7.61-7.57 (m, 1H), 7.31 -7.29 (m, 1H), 6.79-6.77 (m, 1H), 4.74-4.69 (m, 1H), 3.57 (s, 3H), 2.67-2.53 (m, 2H), 2.13-2.01 (m, 5H). ee %=100%.

Compound B was found to have an IP₅₀ of 3.6 nM against BRM and 5.7 nM against BRG1 in the ATPase assay described.

### Example 8. Effects of BRG1/BRM ATPase Inhibition on the Growth of Uveal Melanoma, Hematological Cancer, Prostate Cancer, Breast Cancer, and Ewing's Sarcoma Cell Lines

Procedure: All cell lines described above in Example 5 were also tested as described above with Compound B. In addition, the following cell lines were also tested as follows. Briefly, for Ewing's sarcoma cell lines (CADOES1, RDES, SKES1), retinoblastoma cell lines (WERIRB1), ALL cell lines (REH), AML cell lines (KASUMI1), prostate cancer cell lines (PC3, DU145, 22RV1), melanoma cell lines (SH4, SKMEL28, VVM115, COLO829, SKMEL3, A375), breast cancer cell lines (MDAMB415, CAMA1, MCF7, BT474, HCC1419, DU4475, BT549), B-ALL cell lines (SUPB15), CML cell lines (K562, MEG01), Burkitt's lymphoma cell lines (RAMOS2G64C10, DAUDI), mantle cell lymphoma cell lines (JEKO1, REC1), bladder cancer cell lines (HT1197), and lung cancer cell lines (SBC5), the above methods were performed with the following modifications: Cells were plated in 96 well plates, and the next day, BRG1/BRM ATPase inhibitor, Compound B, was dissolved in DMSO and added to the cells in a concentration gradient from 0 to 10 μM. At the time of cell splitting on days 3 and 7, cells were split into new 96 well plates, and fresh compound was added four hours after re-plating. Table 5 lists the tested cell lines and growth media used.

TABLE 5
Cell Lines And Growth Media

| Cell Line | Source | Growth Media |
|---|---|---|
| 22RV1 | ATCC | RPMI1640 + 10% FBS |
| A375 | ATCC | DMEM + 10% FBS |
| BT474 | ATCC | Hybricare medium + 1.5 g/L sodium |
| | | bicarbonate + 10% FBS |
| BT549 | ATCC | RPMI1640 + 0.023 IU/ml insulin + |
| | | 10% FBS |
| CADOES1 | DSMZ | RPMI1640 + 10% FBS |
| CAMA1 | ATCC | EMEM + 10% FBS |
| COLO829 | ATCC | RPMI1640 + 10% FBS |
| DAUDI | ATCC | RPMI1640 + 10% FBS |
| DU145 | ATCC | EMEM + 10% FBS |
| DU4475 | ATCC | RPMI1640 + 10% FBS |
| HCC1419 | ATCC | RPMI1640 + 10% FBS |
| HT1197 | ATCC | EMEM + 10% FBS |
| JEKO1 | ATCC | RPMI1640 + 20% FBS |
| K562 | ATCC | IMDM + 10% FBS |
| KASUMI1 | ATCC | RPMI1640 + 10% FBS |
| MCF7 | ATCC | EMEM + 0.01 mg/ml bovine insulin + |
| | | 10% FBS |
| MDAMB415 | ATCC | Leibovitz's L-15 + 2 mM L-glutamine + |
| | | 10 mcg/ml insulin + 10 mcg/ml |
| | | glutathione + 15% FBS |
| MEG01 | ATCC | RPMI1640 + 10% FBS |
| PC3 | ATCC | F-12K + 10% FBS |
| RAMOS2G64C10 | ATCC | RPMI1640 + 10% FBS |
| RDES | ATCC | RPMI1640 + 15% FBS |
| REC1 | ATCC | RPMI1640 + 10% FBS |
| REH | ATCC | RPMI1640 + 10% FBS |
| SBC5 | JCRB | EMEM + 10% FBS |
| SH4 | ATCC | DMEM + 10% FBS |
| SKES1 | ATCC | McCoy's 5A + 15% FBS |
| SKMEL28 | ATCC | EMEM + 10% FBS |
| SKMEL3 | ATCC | McCoy's 5A + 15% FBS |
| SUPB15 | ATCC | IMDM + 4 mM L-glutamine + 1.5 g/L |
| | | sodium bicarbonate + 0.05 mM 2- |
| | | mercaptoethanol + 20% FBS |
| WERIRB1 | ATCC | RPMI1640 + 10% FBS |
| WM115 | ATCC | EMEM + 10% FBS |

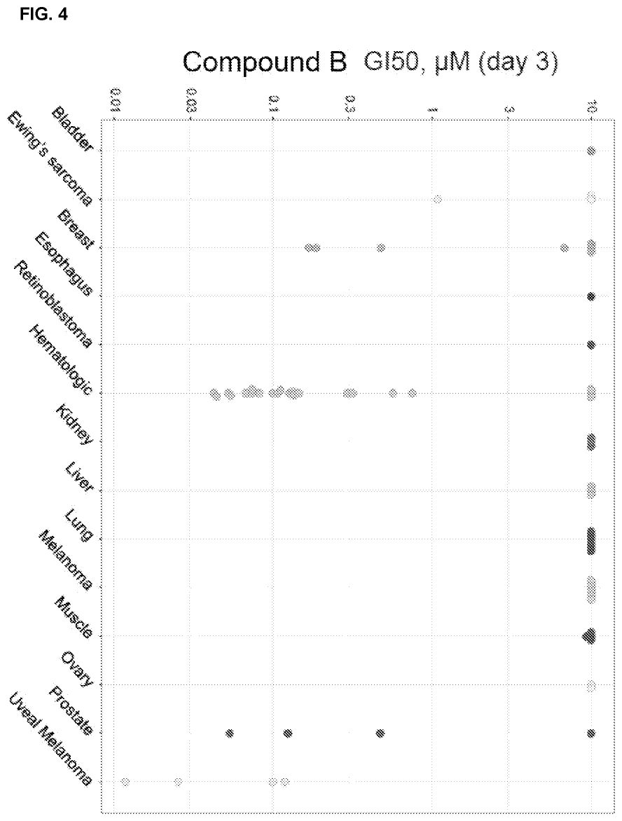

Results: As shown in FIG. 4, the uveal melanoma, hematologic cancer, prostate cancer, breast cancer, and Ewing's sarcoma cell lines were more sensitive to BRG1/BRM inhibition than the other tested cell lines. Inhibition of the uveal melanoma, hematologic cancer, prostate cancer, breast cancer, and Ewing's sarcoma cell lines was maintained through day 7.

### Example 9. Effects of BRG1/BRM ATPase inhibition on the growth of cancer cell lines.

Procedure: A pooled cell viability assay was performed using PRISM (Profiling Relative Inhibition

Simultaneously in Mixtures) as previously described (“High-throughput identification of genotype-specific cancer vulnerabilities in mixtures of barcoded tumor cell lines”, Yu et al, Nature Biotechnology 34, 419-423, 2016), with the following modifications. Cell lines were obtained from the Cancer Cell Line Encyclopedia (CCLE) collection and adapted to RPMI-1640 medium without phenol red, supplemented with 10% heat-inactivated fetal bovine serum (FBS), in order to apply a unique infection and pooling protocol to such a big compendium of cell lines. A lentiviral spin-infection protocol was executed to introduce a 24 nucleotide-barcode in each cell line, with an estimated multiplicity of infection (MOI) of 1 for all cell lines, using blasticidin as selection marker. Over 750 PRISM cancer cell lines stably barcoded were then pooled together according to doubling time in pools of 25. For the screen execution, instead of plating a pool of 25 cell lines in each well as previously described (Yu et al.), all the adherent or all the suspension cell line pools were plated together using T25 flasks (100,000 cells/flask) or 6-well plates (50,000 cells/well), respectively. Cells were treated with either DMSO or compound in a 8-point 3-fold dose response in triplicate, starting from a top concentration of 10 μM. As control for assay robustness, cells were treated in parallel with two previously validated compounds, the pan-Raf inhibitor AZ-628, and the proteasome inhibitor bortezomib, using a top concentration of 2.5 μM and 0.039 μM, respectively.

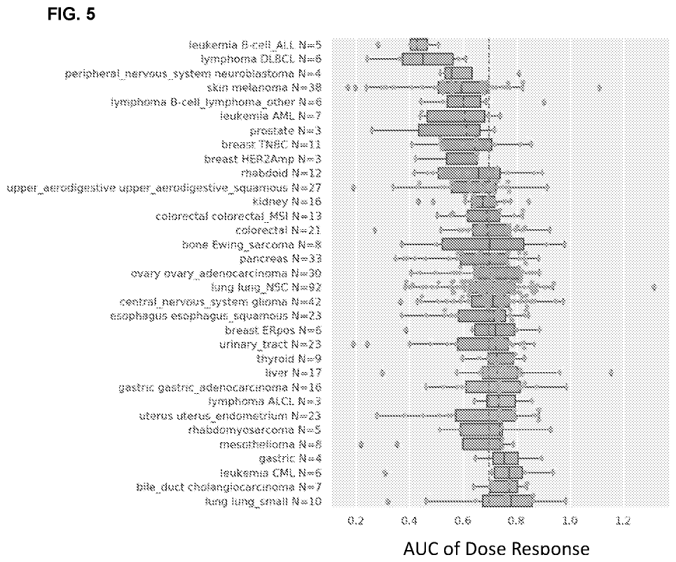

Following 3 days of treatment with compounds, cells were lysed, genomic DNA was extracted, barcodes were amplified by PCR and detected with Next-Generation Sequencing. Cell viability was determined by comparing the counts of cell-line specific barcodes in treated samples to those in the DMSO-control and Day 0 control. Dose-response curves were fit for each cell line and corresponding area under the curves (AUCs) were calculated and compared to the median AUC of all cell lines (FIG. 5).

Results: Cell lines with AUCs less than the median were considered most sensitive.

### Example 10. Effects of BRG1/BRM ATPase Inhibitors on the Growth of Uveal Melanoma Cell Lines

Procedure: Uveal melanoma cell lines (92-1, MP41, MP38, MP46) and non-small cell lung cancer cells (NCIH1299) were plated into 96 well plates with growth media (see Table 5). BRG1/BRM

ATPase inhibitor, Compound B, was dissolved in DMSO and added to the cells in a concentration gradient from 0 to 10 μM at the time of plating. Cells were incubated at 37° C. for 3 days. After three days of treatment, cell growth was measured with Cell-titer glow (Promega), and luminescence was read on an Envision plate reader (Perkin Elmer).

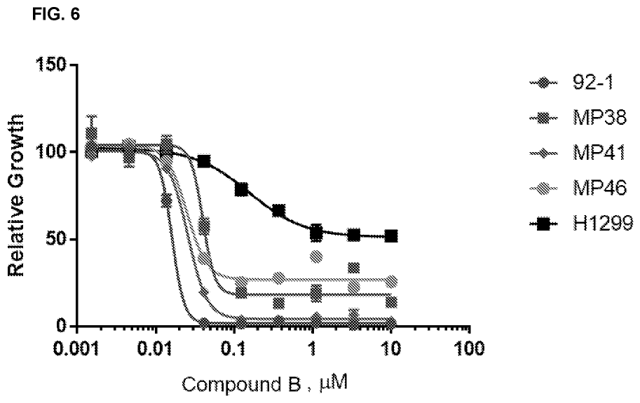

Results: As shown in FIG. 6, Compound B resulted in potent growth inhibition in the cell lines.

### Example 11. Comparison of BRG1/BRM Inhibitors to Clinical PKC and MEK Inhibitors in Uveal Melanoma Cell Lines

Procedure: Uveal melanoma cell lines, 92-1 or MP41, were plated in 96 well plates in the presence of growth media (see Table 5). BAF ATPase inhibitor (Compound B), PKC inhibitor (LXS196; MedChemExpress), and MEK inhibitor (selumetinib; Selleck Chemicals) were dissolved in DMSO and added to the cells in a concentration gradient from 0 to 10 μM at the time of plating. Cells were incubated at 37° C. for 3 days. After three days of treatment, cell growth was measured with Cell-titer glow (Promega), and luminescence was read on an Envision plate reader (Perkin Elmer).

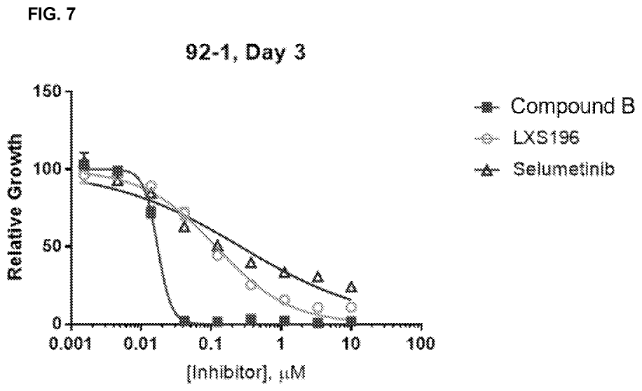

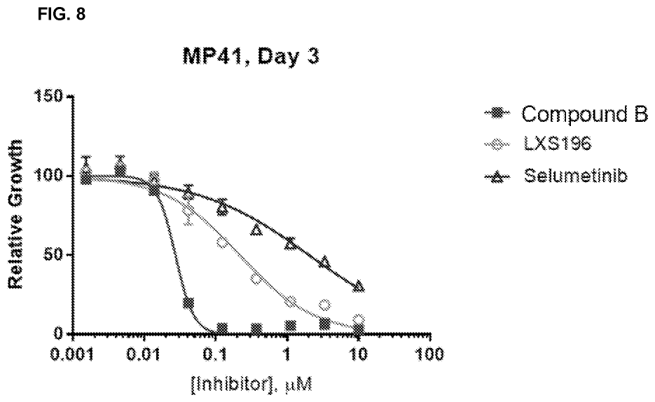

Results: As shown in FIG. 7 and FIG. 8, Compound B showed more potent effects on growth inhibition of uveal melanoma cells as compared to the clinical PKC and MEK inhibitors. Further, Compound B was found to result in a faster onset of growth inhibition than the clinical PKC and MEK inhibitors.

### Example 12. BRG1/BRM ATPase inhibitors are Effective at Inhibiting the Growth of PKC Inhibitor-resistant Cells

Procedure: MP41 uveal melanoma cells were made resistant to the PKC inhibitor (LXS196; MedChemExpress), by long-term culture in growth media (see Table 5) containing increasing concentrations of the. compound, up to 1 μM. After 3 months, sensitivity of the parental MP41 cells and the PKC inhibitor (PKCl)-resistant cells to the PKC inhibitor (LXS196) or the BRG1/BRM ATPase inhibitor (Compound B) was tested in a 7-day growth inhibition assay as described above in Example 4.

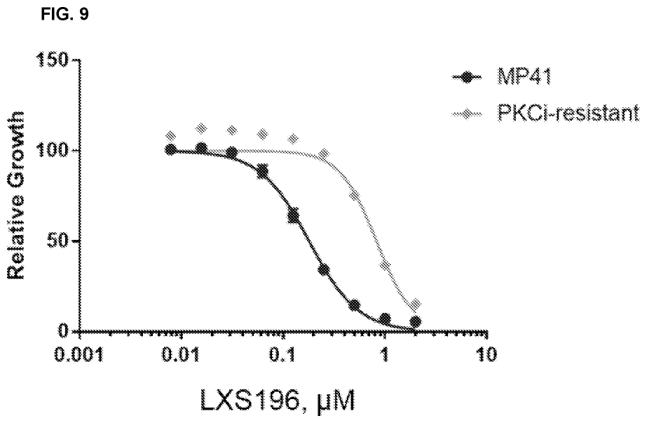

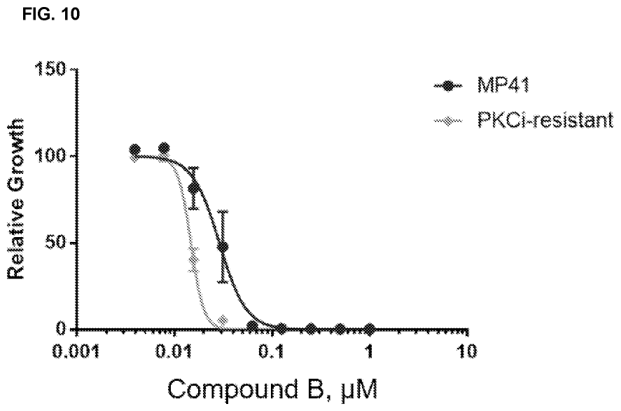

Results: While the PKCl-resistant cells could tolerate growth at higher concentrations of LXS196 than could the parental MP41 cell line (FIG. 9), the BRG1/BRM ATPase inhibitor (Compound B) resulted in strong growth inhibition of both the PKCl-resistant and parental cell lines (FIG. 10). The PKCl-resistant cells were more sensitive to Compound B than were the parental MP41 cells (FIG. 10).

### Example 13. Synthesis of Compound C

BRG1/BRM inhibitor Compound C has the structure:

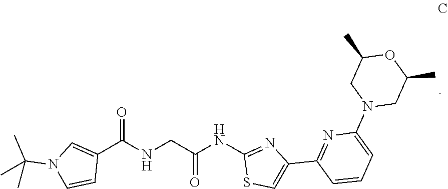 [SMILES: C[C@@H]1CN(c2cccc(-c3csc(NC(=O)CNC(=O)c4ccn(C(C)(C)C)c4)n3)n2)C[C@H](C)O1]

Compound C was synthesized as shown in Scheme 5 below.

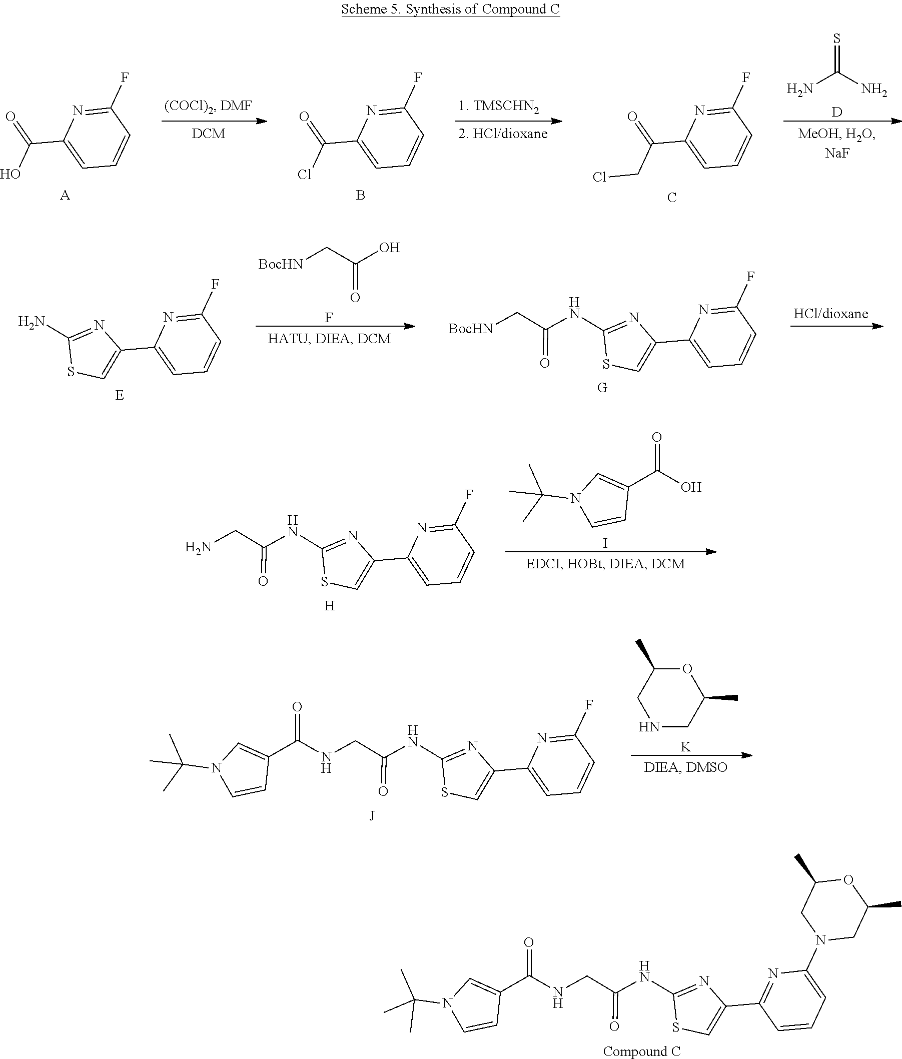

Compound C was found to have an IP₅₀ of 5.3 nM against BRM and 1.3 nM against BRG1 in the ATPase assay described above.

### Example 14. BRG1/BRM ATPase Inhibitors Cause Uveal Melanoma Tumor Growth Inhibition In Vivo.

Procedure: Nude mice (Envigo) were engrafted subcutaneously in the axillary region with 5×10⁶ 92-1 uveal melanoma cells in 50% Matrigel. Tumors were grown to a mean of ˜200 mm³, at which point mice were grouped and dosing was initiated. Mice were dosed once daily by oral gavage with vehicle (20% 2-Hydroxypropyl-β-Cyclodextrin) or increasing doses of Compound C. Tumor volumes and body weights were measured over the course of 3 weeks, and doses were adjusted by body weight to achieve the proper dose in terms of mg/kg. At this time, animals were sacrificed, and tumors were dissected and imaged.

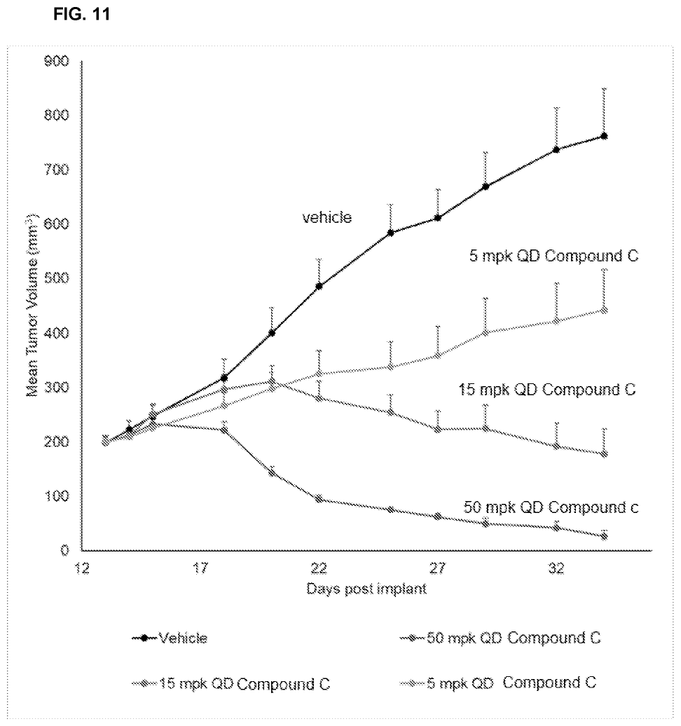

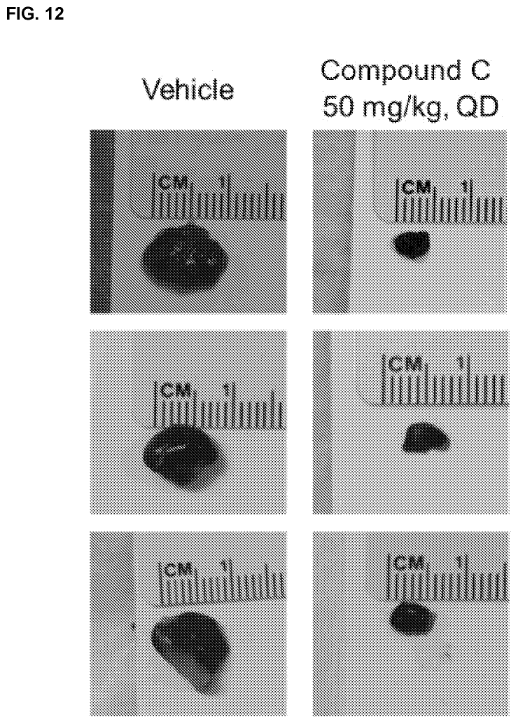

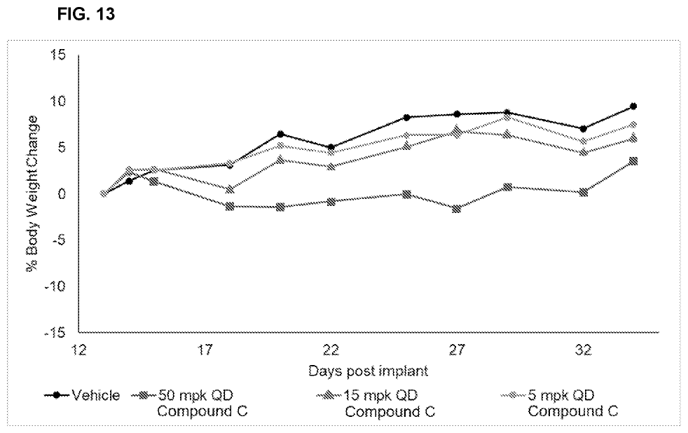

Results: As shown in FIG. 11 and FIG. 12, treatment with Compound C led to tumor growth inhibition in a dose-dependent manner with tumor regression observed at the highest (50 mg/kg) dose. As shown in FIG. 13, all treatments were well tolerated with no body weight loss observed (FIG. 13).

### Example 15. Effects of BRG1/BRM ATPase Inhibition on the Growth of Uveal Melanoma and Hematological Cancer Cell Lines

Procedure: Uveal melanoma cell lines (92-1, MEL202, MP41, MP38, MP46), prostate cancer cells (22RV1), acute leukemia cells (EOL1, THP1), and histocytic lymphoma cells (U937) were plated into 96 well plates with growth media (see Table 5). BRG1/BRM ATPase inhibitor, N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1H-pyrrole-3-carboxamide, was dissolved in DMSO and added to the cells in a concentration gradient from 0 to 2 μM (for uveal melanoma cell lines), or 0 to 1 μM (for other cell lines), at the time of plating. Cells were incubated at 37° C. for 3 days. After three days of treatment, cell growth was measured with Cell-titer glow (Promega), and luminescence was read on an Envision plate reader (Perkin Elmer).

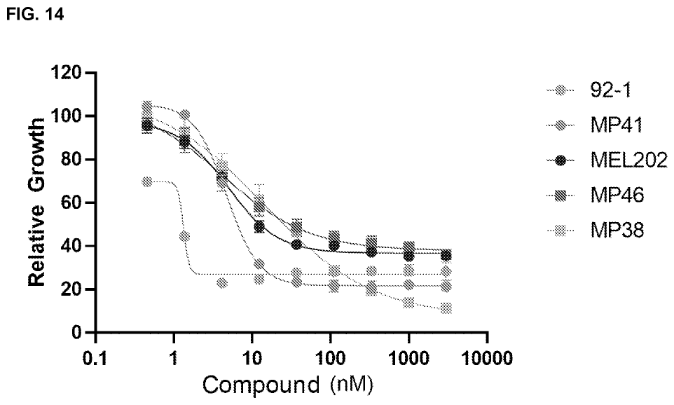

Results: As shown in FIG. 14, N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1H- pyrrole-3-carboxamide resulted in potent growth inhibition in all the cell lines. As shown in Table 6, measured absolute IC₅₀ values were below 350 nanomolar for all cell lines tested.

Table 6 lists the tested cell lines, growth media used, and absolute IC₅₀ values (nM) after 3 days of compound treatment.

TABLE 6
Cell Lines, Growth Media, and Absolute IC₅₀ values

| | | | | Absolute |
|---|---|---|---|---|
| Cell Line | Source | Growth Media | Cancer Type | IC₅₀ (nM) |
| 22RV1 | ATCC | RPMI1640 + 10% | Prostate | 29.7 |
| | | FBS | | |
| 92-1 | SIGMA | RPMI1640 + 10% | Uveal melanoma | 0.3 |
| | | FBS | | |
| EOL1 | DSMZ | RPMI1640 + 10% | Acute myeloid | 75.5 |
| | | FBS | leukemia | |
| MEL202 | SIGMA | RPMI1640 + 10% | Uveal melanoma | 62.3 |
| | | FBS | | |
| MP38 | ATCC | RPMI1640 + 20% | Uveal melanoma | 31.5 |
| | | FBS | | |
| MP41 | ATCC | RPMI1640 + 20% | Uveal melanoma | 11.8 |
| | | FBS | | |
| MP46 | ATCC | RPMI1640 + 20% | Uveal melanoma | 112.6 |
| | | FBS | | |
| THP1 | ATCC | RPMI1640 + 10% | Acute monocytic | 344.9 |
| | | FBS | leukemia | |
| U937 | ATCC | RPMI1640 + 10% | Histiocytic | 14.8 |
| | | FBS | lymphoma | |

### Example 16. BRG1/BRM ATPase Inhibition Causes Uveal Melanoma Tumor Growth Inhibition In Vivo

Procedure: Nude mice (Envigo) were engrafted subcutaneously in the axillary region with 5×10⁶ 92-1 uveal melanoma cells in 50% Matrigel. Tumors were grown to a mean of ˜200 mm³, at which point mice were grouped and dosing was initiated. Mice were dosed once daily by oral gavage with vehicle (20% 2-Hydroxypropyl-β-Cyclodextrin) or increasing doses of N-((S)-1-((4-(6-(cis-2,6-dimethylmorpholino)pyridin-2-yl)thiazol-2-yl)amino)-3-methoxy-1-oxopropan-2-yl)-1-(methylsulfonyl)-1 H-pyrrole-3-carboxamide. Tumor volumes and body weights were measured over the course of 3 weeks, and doses were adjusted by body weight to achieve the proper dose in terms of mg/kg.

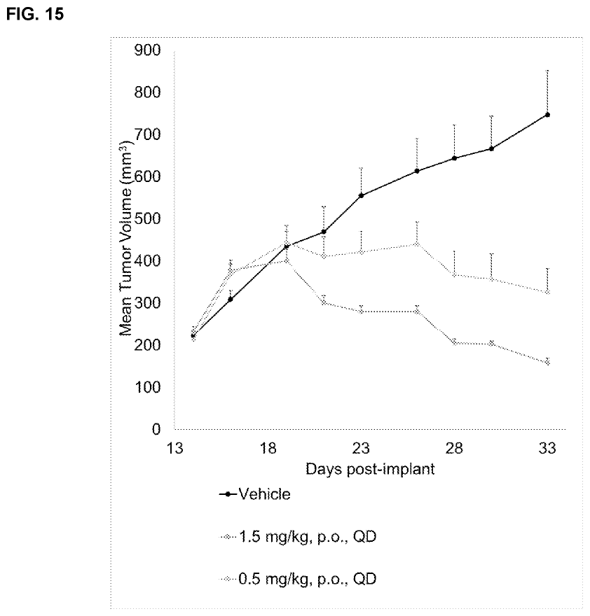

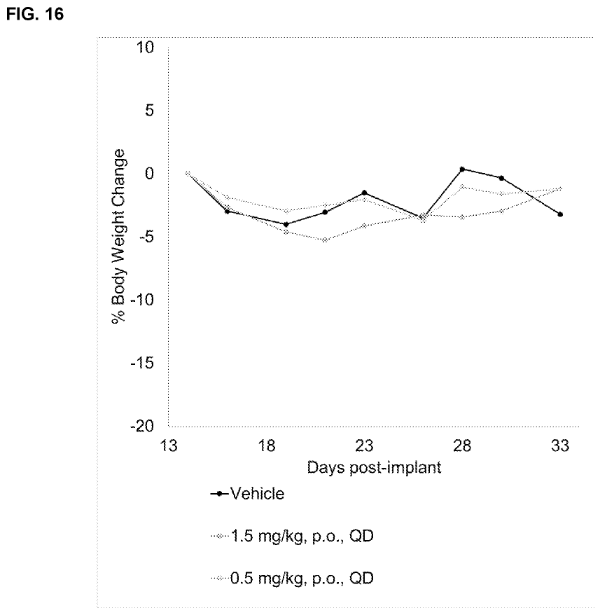

Results: As shown in FIG. 15, treatment with N-((S)-1-((4-(6-(cis-2,6-dimethylmorpholino)pyridin-2-yl)thiazol-2-yl)amino)-3-methoxy-1-oxopropan-2-yl)-1- methylsulfonyl)-1 H-pyrrole-3-carboxamide led to tumor growth inhibition in a dose-dependent manner with tumor regression observed at the highest (1.5 mg/kg) dose. As shown in FIG. 16, all treatments were well tolerated based on % body weight change observed.

### Example 17. In-vitro Profiling of P-glycoprotein (P-gp) and Breast Cancer Resistance Protein (BCRP) and Other Transporters

In vitro membrane permeability. In vitro bidirectional transport studies were conducted to determine passive permeability, and P-gp and BCRP efflux status of the compound of formula (I) using MDCK subclone II (MDCK-II) cells expressing human BCRP or P-gp.

The compound of formula (I) showed concentration-dependent efflux in MDCK-II cells expressing BCRP or P-gp, but not in MDCK control cells (Table 7). Efflux ratio (ER) of the compound of formula (I) at 1 μM in the BCRP expressing cells was reduced from 4 to 0.9 in the presence of 1 μM Ko143, a BCRP inhibitor. The ER of the compound of formula (I) in the P-gp expressing cells at 1 μM was reduced from 16 to 0.8 in the presence of 3 μM elacridar, a P-gp inhibitor. In the MDCK control cells, the compound of formula (I) showed apparent permeability coefficient (Pₐₚₚ) (B→A) or Pₐₚₚ (A→B) of ≥15 (10⁻⁶ cm/s) These data indicate that the compound of formula (I) is a substrate for both BCRP and P-gp and has high passive permeability.

TABLE 7

| | | In transporter cells | | | In control cells | | |
|---|---|---|---|---|---|---|---|
| | Assay | Pₐₚₚ (×10⁻⁶ cm/s) | | | Pₐₚₚ (×10⁻⁶ cm/s) | | |
| Transporter | Condition | B→A | A→B | ER | B→A | A→B | ER |
| BCRP | 1 μM | 24.3 | 6.15 | 3.95 | 18.2 | 26.0 | 0.701 |
| | compound of formula (I) | | | | | | |
| | 1 μM | 24.6 | 27.2 | 0.906 | 17.9 | 31.6 | 0.566 |
| | compound of formula (I) + | | | | | | |
| | 1 μM Ko143 ^{a} | | | | | | |
| | 10 μM | 25.2 | 16.5 | 1.53 | 20.9 | 25.4 | 0.823 |
| | compound of formula (I) | | | | | | |
| | 50 μM | 18.5 | 22.4 | 0.829 | 19.2 | 21.3 | 0.903 |
| | compound of formula (I) | | | | | | |
| P-gp | 1 μM | 27.0 | 1.71 | 15.8 | 21.0 | 28.8 | 0.731 |
| | compound of formula (I) | | | | | | |
| | 1 μM | 23.7 | 31.2 | 0.759 | 17.1 | 33.7 | 0.508 |
| | compound of formula (I) + | | | | | | |
| | 3 μM elacridar ^{a} | | | | | | |
| | 10 μM | 53.1 | 4.62 | 11.5 | 23.6 | 31.0 | 0.761 |
| | compound of formula (I) | | | | | | |
| | 50 μM | 22.8 | 12.6 | 1.81 | 18.5 | 25.5 | 0.728 |
| | compound of formula (I) | | | | | | |

^{a} = Ko143 and elacridar are used as reference inhibitors for each transporter.
ER = Efflux ratio.
Pₐₚₚ = Apparent permeability.

Potential Inhibition of Transporters. The potential for the compound of formula (I) to inhibit a panel of cellular transporters was investigated using polarized monolayer of MDCK-II cells grown on permeable supports for organic cation transporter (OAT) 1, OAT3, organic cation transporter (OCT) 2, OATP1B1, OATP1B3, multidrug and toxin extrusion transporter (MATE) 1, MATE2, BCRP, and P-gp. The compound of formula (I) was tested at various concentrations up to 100 μM for the BCRP and P-gp assays or at various concentrations up to 50 μM for all other transporters.

The compound of formula (I) showed minimal inhibition toward OAT1 and OCT2 at nominal test concentrations up to 50 μM. The compound of formula (I) inhibited the other transporters OAT3, OATP1B1, OATP1 B3, MATE1, MATE2-K, BCRP, and P-gp. Estimated IC₅₀s ranged from 1.35 μM for MATE1 to 24.6 μM for OAT3 using the nominal test concentrations. The IC₅₀ ranged from 0.318 μM for MATE1 to 5.79 μM for OAT3 after being corrected for non-specific binding (the average difference between measured and nominal concentrations; Table 8).

TABLE 8
Estimated IC₅₀s for the compound of formula (I) in
a panel of transporters expressed in MDCK-II cells

| | | Estimated IC₅₀ | IC₅₀ corrected |
|---|---|---|---|
| | | using nominal | for non-specific |
| | | concentrations | binding |
| Transporters | Probe substrate | (μM) | (μM) ^{a} |
| OAT1 | p-aminohippurate | >50 | >11.8 |
| | 2 μM | | |
| OAT3 | estrone-3-sulfate | ~24.6 | ~5.79 |
| | 0.1 μM | | |
| OCT2 | metformin 10 μM | >50 | >11.8 |
| OATP1B1 | estradiol-17-β-D- | 11.4 | 2.68 |
| | glucuronide 2 μM | | |
| OATP1B3 | CCK-8 2 μM | 11.4 | 2.68 |
| MATE1 | metformin 10 μM | 1.35 | 0.318 |
| MATE2-K | metformin 10 μM | 9.73 | 2.29 |
| BCRP | prazosin 2 μM | 10.1 | 2.38 |
| P-gp | quinidine 0.1 μM | 18.5 | 4.36 |

^{a} = Corrected for the average difference between measured and nominal concentrations in the OCT2 assay.

### Example 18. Identification of Human Cytochrome P450 Enzymes Involved in Metabolism of the Compound of Formula (I)

A reaction phenotyping study was conducted to evaluate the role of cytochrome P450 (CYP) enzymes in compound of formula (I) in vitro metabolism. Based on the selective chemical inhibition of compound of formula (I) depletion in human liver microsomes and the rate of depletion exerted by 7 recombinant CYP enzymes (CYP3A4, CYP2D6, CYP2C9, CYP2C19, CYP1A2, CYP2B6, and CYP2C8), CYP3A was identified as the major CYP enzyme responsible for compound of formula (I) metabolism in human liver microsomes with a possible involvement of CYP2C19 to a lesser extent. The results of this study are presented in Tables 9 and 10.

TABLE 9

| | % Remaining of Compound of Formula (I) | | | | | | | | |
|---|---|---|---|---|---|---|---|---|---|
| Compound of Formula | Control | Reductase | | | | | | | |
| (I) Concentration (μM) | Bactosomes | Control | 1A2 | 2B6 | 2C8 | 2C9 | 2C19 | 2D6 | 3A4 |
| 0.1 | 90.2 | 78.3 | 89.4 | 85.3 | 81.0 | 79.9 | 80.5 | 85.0 | 0.0 |
| 1 | 98.3 | 109 | 113 | 104 | 95.4 | 100 | 95.0 | 93.5 | 1.2 |

TABLE 10

| | Targeted | | Inhibitor | |
|---|---|---|---|---|
| Inhibitor | CYP | | Concentration | % |
| Type | enzyme | Inhibitor | (μM) | Inhibition |
| Direct | 2D6 | Quinidine | 5 | No |
| | | | | inhibition |
| | 2A6 | Letrozole | 10 | 9.1 |
| | 3A4/5 | Ketoconazole | 1 | 83.3 |
| Time- | 2B6 | Phencyclidine | 30 | 5.7 |
| dependent | 2D6 | Paroxetine | 5 | No |
| | | | | inhibition |
| | 2E1 | Diethyldithio- | 10 | No |
| | | carbamate | | inhibition |
| | 1A2 | Furafylline | 10 | 1.9 |
| | 3A4/5 | Troleandomycin | 50 | 100 |
| | 2C8 | Gemfibrozil | 100 | No |
| | | glucuronide | | inhibition |
| | 2C9 | Tienilic acid | 20 | 2.4 |
| | 2C19 | Esomeprazole | 10 | 27.1 |

Potential CYP enzyme inhibition. The potential for the compound of formula (I) to inhibit a panel of CYP enzymes was assessed in vitro using pooled human liver microsomes (PHLM).

In PHLM, compound of formula (I) at concentrations up to 30 μM showed minimal to modest direct inhibitory activity (<50% inhibition) toward CYP1A2, CYP2B6, CYP2C9, CYP2C19, CYP2D6, and CYP3A. There was direct inhibition of CYP2C8 with an IC₅₀ value of 20 μM. Preincubation with the compound of formula (I) in the presence of nicotinamide adenine dinucleotide phosphate (NADPH) did not enhance inhibition of the CYPs tested except CYP3A. Compared to the inhibition observed after preincubation in the absence of NADPH, CYP3A4/5 inhibition, as measured by midazolam 1′-hydroxylation and testosterone 6β-hydroxylation, was increased by 30 to 40% after 30 minutes preincubation of compound of formula (I) at 30 μM in the presence of NADPH. At concentrations of ≤10 μM, the effect of preincubation on CYP3A4/5 activity was not apparent (<20% increase in inhibition). These data suggest that the compound of formula (I) is a weak time-dependent inhibitor of CYP3A4/5 (Table 11). Due to the limited solubility, and consideration that time-dependent inhibition was only apparent at 30 μM, maximal inactivation rate and KI of CYP3A4/5 could not be determined.

TABLE 11

| | Compound of | | Enzyme activity (% inhibition) | | |
|---|---|---|---|---|---|
| | Formula (I) | | | 30 Minutes | 30 Minutes |
| | Concentration | | No Pre- | Pre-incubation | Pre-incubation |
| Enzyme | (μM) | Probe Substrate | incubation | without NADPH | with NADPH |
| CYP1A2 | 30 | Phenacetin | 11.4 | 8.5 | 11.8 |
| CYP2B6 | 30 | Efavirenz | 7.6 | −6.0 | −10.0 |
| CYP2C8 | 30 | Amodiaquine | 60.2 | 62.7 | 67.1 |
| CYP2C9 | 30 | Diclofenac | 47.1 | 29.5 | 43.5 |
| CYP2C19 | 30 | S-Mephenytoin | 23.1 | 21.4 | 28.1 |
| CYP2D6 | 30 | Dextromethorphan | 13.2 | 11.5 | 9.6 |
| CYP3A4/5 | 10 | Midazolam | 18.2 | 6.5 | 25.3 |
| CYP3A4/5 | 30 | Midazolam | 13.6 | −1.0 | 40.2 |
| CYP3A4/5 | 10 | Testosterone | 9.7 | 10.5 | 27.4 |
| CYP3A4/5 | 30 | Testosterone | 18.4 | 20.8 | 49.3 |

Potential CYP enzyme induction. Two in vitro studies were conducted to evaluate the compound of formula (I) as a CYP inducer using cryopreserved human hepatocytes from 3 donors for each study. The positive controls and the negative control performed as expected in both studies. For the first study, mRNA levels of CYP1A2, CYP2B6 and CYP3A4 were measured by qRT-PCR in cultured human hepatocytes treated with the compound of formula (I) for 72 hours at concentrations ranging from 0.05 to 30 μM. The compound of formula (I) at concentrations ≥0.5 μM appeared cytotoxic to cultured human hepatocytes. Morphology changes of hepatocytes were observed at compound of formula (I) concentrations ≥0.5 μM for all donors, though increased LDH release was only observed in the culture of one donor (HC10-23). CYP1A2 and CYP3A4 expressions were profoundly decreased by the compound of formula (I) at concentrations of 0.05 and 0.15 μM that showed no apparent effect on hepatocyte morphology. CYP2B6 mRNA level was also significantly decreased at 0.15 μM, but not at 0.05 μM (Table 12).

TABLE 12

| Compound of | | | | |
|---|---|---|---|---|
| Formula (I) Nominal | | | | |
| Concentration | CYP | mRNA Fold Change Relative to DMSO Control ± SD | | |
| (μM) | Isoform | HC10-23 | HC10-50 | HC5-27 |
| 0.05 | CYP1A2 | 0.0703 ± 0.0064 | 0.0567 ± 0.0025 | 0.044 ± 0.0036 |
| | CYP2B6 | 1.29 ± 0.21 | 0.877 ± 0.127 | 0.381 ± 0.029 |
| | CYP3A4 | 0.001 ± 0.000 | 0.006 ± 0.001 | 0.00233 ± 0.00153 |
| 0.15 | CYP1A2 | N/A | 0.0127 ± 0.0006 | N/A |
| | CYP2B6 | 0.258 ± 0.021 | 0.300 ± 0.05 | 0.222 ± 0.016 |
| | CYP3A4 | N/A | 0.007 ± 0.001 | 0.001 |

N/A = Not applicable due to insufficient RNA or signals below the limits of quantitation.
SD = Standard deviation.
Values are means ± standard deviation of triplicate determinations.

The second study was conducted to further assess the effects of the compound of formula (I) on CYP mRNA expressions or enzyme activities in cultured cryopreserved human hepatocytes treated with the compound of formula (I) for 72 hours at lower test concentrations (0.3 to 50 nM) (Table 13). There was no evidence of cytotoxicity at the tested concentrations. Consistent with the first study, compound of formula (I) treatments decreased mRNA levels of CYP1A2 and CYP3A4 in a concentration-dependent manner. Expression of mRNA for CYP2C8 and CYP2C9, but not CYP2B6 and CYP2C19, was also down-regulated. More than a 50% decrease in mRNA levels was observed at compound of formula (I) concentrations nM for CYP3A4 and 0 nM for CYP1A2, CYP2C8 and CYP2C9. Consistent with mRNA expression, CYP1A2 enzyme activity was decreased by >50% at compound of formula (I) concentrations ≥10 nM and CYP2B6 activity was not affected (Table 14). In contrast, the effect of the compound of formula (I) on CYP3A activity was minimal, even though CYP3A4 mRNA levels decreased significantly at concentrations ranging from 1 to 50 nM.

TABLE 13

| | | | | | | | Positive Control | |
|---|---|---|---|---|---|---|---|---|
| | Compound of Formula (I) Concentrations (nM)/mRNA | | | | | | | mRNA |
| | Fold Change Relative to the Vehicle Control | | | | | | Compound | Fold |
| Isoforms | 0.3 | 1 | 3 | 10 | 30 | 50 | (μM) | Change* |
| CYP1A2 | 1.12 ± 0.21 | 0.95 ± 0.09 | 0.68 ± 0.05 | 0.41 ± 0.08 | 0.26 ± 0.05 | 0.21 ± 0.03 | Omeprazole (50) | 50.2-118 |
| CYP2B6 | 1.78 ± 0.24 | 1.52 ± 0.47 | 1.46 ± 0.11 | 1.71 ± 0.40 | 1.20 ± 0.08 | 0.97 ± 0.03 | Phenobarbital (1000) | 15.3-39.6 |
| CYP3A4 | 0.87 ± 0.10 | 0.35 ± 0.05 | 0.09 ± 0.05 | 0.03 ± 0.01 | 0.03 ± 0.00 | 0.02 ± 0.00 | Rifampin (20) | 34.0-54.4 |
| CYP2C8 | 0.93 ± 0.21 | 0.92 ± 0.17 | 0.78 ± 0.19 | 0.42 ± 0.15 | 0.29 ± 0.06 | 0.28 ± 0.13 | Rifampin (20) | 3.74-12.5 |
| CYP2C9 | 1.04 ± 0.22 | 1.03 ± 0.17 | 0.93 ± 0.36 | 0.42 ± 0.07 | 0.30 ± 0.05 | 0.20 ± 0.03 | Rifampin (20) | 1.87-4.92 |
| CYP2C19 | 1.11 ± 0.03 | 1.14 ± 0.25 | 1.41 ± 0.52 | 1.04 ± 0.14 | 1.10 ± 0.19 | 0.98 ± 0.32 | Rifampin (20) | 0.61-1.01 |

Values are means ± SD of three donors; * range of fold change observed from three donors. mRNA fold change ranged from 0.841 to 3.19 for the negative control flumazenil (25 μM).

TABLE 14

| | | | | | | | Positive Control | |
|---|---|---|---|---|---|---|---|---|
| | Compound of Formula (I) Concentrations (nM)/Enzyme | | | | | | | Enzyme |
| | Activity Fold Change Relative to the Vehicle Control | | | | | | Compound | Activity |
| Isoforms | 0.3 | 1 | 3 | 10 | 30 | 50 | (μM) | Fold Change |
| CYP1A2 | 0.87 ± 0.08 | 0.81 + 0.07 | 0.92 ± 0.42 | 0.35 ± 0.16 | 0.24 ± 0.12 | 0.21 ± 0.04 | Omeprazole (50) | 20.0-93.9 |
| CYP2B6 | 1.12 ± 0.09 | 1.08 + 0.06 | 1.17 ± 0.02 | 1.09 ± 0.15 | 0.94 ± 0.08 | 0.80 ± 0.15 | Phenobarbital (1000) | 11.9-16.7 |
| CYP3A4 | 0.93 ± 0.15 | 0.86 + 0.19 | 0.73 ± 0.18 | 0.68 ± 0.16 | 0.80 ± 0.28 | 0.90 ± 0.28 | Rifampin (20) | 5.53-29.8 |

Values are means ± SD of three donors; * range of fold change observed from three donors. Enzyme activity fold change ranged from 0.636 to 1.55 for the negative control flumazenil (25 μM).

The compound of formula (I) was also evaluated in rat hepatocytes (0.3 to 100 nM) to determine if CYP downregulation occurs in this species. In cultured rat hepatocytes, mRNA expressions of CYP2b1 and CYP3a23, but not CYP1a2, were decreased ≥50% by the compound of formula (I) at concentrations ≥10 nM.

Pharmacokinetic drug interactions. No specific studies have been conducted to evaluate potential interactions with drugs that may be co-administered with the compound of formula (I). In PHLM, the IC₅₀ value of direct inhibition was 20 μM for CYP2C8 and >30 μM for CYP1A2, CYP2B6, CYP2C9, CYP2C19, CYP2D6, and CYP3A4/5. The compound of formula (I) was a weak time-dependent inhibitor of CYP3A4/5. In cultured human hepatocytes, compound of formula (I) treatments down-regulated mRNA expression of CYP3A4 at concentrations ≥1 nM, and CYP1A2, CYP2C8 and CYP2C9 at concentrations ≥10 nM, but had minimal effect on mRNA expression of CYP2B6 and CYP2C19 at the tested concentrations up to 50 nM. In cells over-expressing specific transporters, the compound of formula (I) inhibited OAT3, OATP1B1, OATP1 B3, MATE1, MATE2-K, BCRP and P-gp, but not OAT1 and OCT2. The estimated IC₅₀ concentrations ranged from 0.318 μM for MATE1 up to 5.79 μM for OAT3 after being corrected for non-specific binding.

Based on a predicted maximum observed plasma concentration (Cₘₐₓ) of 0.8 to 1.4 μM at a human dose of 300 mg and Kₐ of 0.1 h⁻¹ and assuming fᵤ of 0.01 in human plasma, R-values (calculated according to the FDA Guidance for Industry “In Vitro Drug Interaction Studies—Cytochrome P450 Enzyme- and Transporter-Mediated Drug Interactions”, January 2020) were <1.02 for all the CYPs tested, <1.1 for OATP1B1 and OATP1B3, and <0.1 for OAT1, OAT3, OCT2, MATE1 and MATE2-K. R-values for P-gp and BCRP inhibition in the gut were >10. These data suggest that the compound of formula (I) has low potential of drug-drug interaction (DDI) as an inhibitor of CYP enzymes and transporters except for P-gp and BCRP. P-gp and BCRP inhibition in the gut may be clinically relevant for sensitive P-gp and BCRP substrates that are orally administered and have a narrow therapeutic index.

In cultured human hepatocytes treated with the compound of formula (I), mRNA levels were decreased >50% at compound of formula (I) concentrations nM for CYP3A4 and ≥10 nM for

CYP1A2, CYP2C8, and CYP2C9. Expression of mRNA for CYP2B6 and CYP2C19 was not affected (mRNA fold changes relative to the vehicle control ranged from ˜1 to 1.78) at concentrations up to 50 nM. Though CYP3A4 mRNA levels decreased at concentrations 1 to 50 nM, CYP3A activity was not significantly affected (≤32% decrease). CYP1A2 enzyme activity was decreased by >50% at compound of formula (I) concentrations ≥10 nM. Since in vitro to in vivo extrapolation of CYP down-regulation has not been established (see 2020 FDA in vitro DDI guidance and Hariparsad et al. (Drug Metabolism and Disposition. 45(10):1049-1059 (2017), which is incorporated herein by reference), the in vivo relevance of these findings remains to be determined. The compound of formula (I) is a P-gp and BCRP substrate with good passive permeability. Considering its high passive membrane permeability and good oral absorption in dogs when administered in a capsule, oral absorption of the compound of formula (I) in humans may not be limited by P-gp and BCRP significantly. As CYP3A was identified as the major CYP enzyme responsible for compound of formula (I) in vitro metabolism, coadministration with strong CYP3A inhibitors or inducers may alter the PK of the compound of formula (I).

As CYP3A was identified as the major CYP enzyme responsible for metabolism of the compound of formula (I) in human liver microsomes and the clinical relevance of CYP down-regulation is unknown, coadministration with strong CYP3A inhibitors or inducers and sensitive CYP3A substrates with a narrow therapeutic index should be avoided until relevant clinical data become available.

### Example 19. Treatment of a BAF Complex-related Disorder in a Human Subject by Administration of the Compound of Formula (I)

The compound of formula (I) can be administered to a human subject in order to treat a BAF complex-related disorder (or decrease the activity of a BAF complex in a subject), such as, e.g., a cancer (such as, e.g., uveal melanoma, advanced hematologic malignancy), a viral infection, coffin siris, neurofibromatosis, or multiple meningioma. For instance, a human subject suffering from uveal melanoma can be treated by administering the compound of formula (I) by an appropriate route (e.g., orally) at a particular dosage (e.g., starting at 5 mg daily) over a course of days, weeks, or months.

A subject should not be administered the compound of formula (I) if the subject is receiving treatment with a known strong CYP3A (e.g., CYP3A4) inhibitor(s) (e.g., boceprevir, cobicistat, danoprevir, elvitegravir, grapefruit juice, indinavir, itraconazole, ketoconazole, lopinavir, paritaprevir, ombitasvir, dasabuvir, posaconazole, ritonavir, saquinavir, telaprevir, tipranavir, telithromycin, troleandomycin, voriconazole, clarithromycin, idelalisib, nefazodone, nelfinavir, or a pharmaceutically acceptable salt thereof), strong CYP3A inducer(s) (e.g., apalutamide, carbamazepine, enzalutamide, mitotane, phenytoin, rifampin, St. John's wort, or a pharmaceutically acceptable salt thereof), or sensitive CYP3A substrate(s) with narrow therapeutic indices (Tis) (e.g., alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, tolvaptan, or a pharmaceutically acceptable salt thereof) that cannot be discontinued prior to compound of formula (I)-administration. A subject should be administered the compound of formula (I) with caution (e.g., with careful monitoring) if the subject is receiving treatment with a known moderate CYP3A inhibitor(s) (e.g., aprepitant, ciprofloxacin, conivaptan, crizotinib, cyclosporine, diltiazem, dronedarone, erythromycin, fluconazole, fluvoxamine, imatinib, tofisopam, verapamil, or a pharmaceutically acceptable salt thereof), moderate CYP3A inducer(s) (e.g., bosentan, efavirenz, etravirine, phenobarbital, primidone, or a pharmaceutically acceptable salt thereof), or substrate(s) that is(are) predominantly metabolized by CYP3A, CYP1A2, CYP2C8, and CYP2C9. It may be necessary to reduce (e.g., lower the dose)/suspend administration of the moderate CYP3A inhibitor(s), moderate CYP3A inducer(s), or substrate(s) that is(are) predominantly metabolized by CYP3A, CYP1A2, CYP2C8, and CYP2C9 or reduce (e.g., lower the dose)/suspend administration of the compound of formula (I).

A subject should not be administered the compound of formula (I) if the subject is receiving treatment with an orally administered medication(s) known to have narrow Tls that are sensitive P-glycoprotein (P-gp) substrate(s) (such as, e.g., dabigatran etexilate, digoxin, fexofenadine, loperamide, quinidine, talinolol, vinblastine, or a pharmaceutically acceptable salt thereof) or breast cancer resistance protein (BCRP) substrate(s) (such as, e.g., coumestrol, daidzein, dantrolene, estrone-3-sulfate, genistein, prazosin, sulfasalazine, rosuvastatin, or a pharmaceutically acceptable salt thereof) that cannot be discontinued prior to compound of formula (I)-administration.

A subject should not be administered the compound of formula (I) if the subject is receiving treatment with a medication(s) that is(are) known to be acid-reducing agents (ARAs (such as, e.g., histamine H2-receptor antagonists (H2 blockers) (e.g., famotidine, cimetidine, ranitidine, nizatidine, or a pharmaceutically acceptable salt thereof), proton pump inhibitors (PPIs) (e.g., omeprazole, lansoprazole, pantoprazole, rabeprazole, esomeprazole, dexlansoprazole, ilaprazole, or a pharmaceutically acceptable salt thereof) that cannot be discontinued prior to compound of formula (I)-administration. Antacids are acceptable when administered in a staggered dosing manner with the compound of formula (I). Under the staggered dosing protocol, the compound of formula (I) is not administered within 2 hours before or after the antacid administration.

A subject should be carefully monitored when treated with the compound of formula (I) if the subject is suffering from thrombocytopenia (e.g., platelets <50×10⁹/L) or another major bleeding disorder/diathesis.

A subject may be carefully monitored when treated with the compound of formula (I) if the subject has or is at possible risk of having QT prolongation.

### Other Embodiments

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A method of providing the compound of formula (I) to a subject in need thereof, the compound of formula (I) having the following structure: 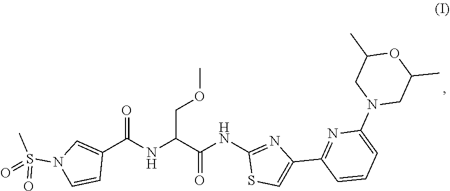 [SMILES: COCC(NC(=O)c1ccn(S(C)(=O)=O)c1)C(=O)Nc1nc(-c2cccc(N3CC(C)OC(C)C3)n2)cs1]
the method comprising administering to the subject a therapeutic regimen comprising the compound of formula (I), or a pharmaceutically acceptable salt thereof, to the subject;
wherein the subject is not administered a CYP3A inhibitor, a CYP3A inducer, a sensitive CYP3A substrate with a narrow therapeutic index, a sensitive P-gp substrate with a narrow therapeutic index, a sensitive BCRP substrate with a narrow therapeutic index, or a combination thereof concomitantly with the regimen.

2. A method of inhibiting cell proliferation in a cancer tissue comprising cancer cells in a subject in need thereof, the method comprising contacting the cancer tissue with the compound of formula (I) according to a therapeutic regimen: 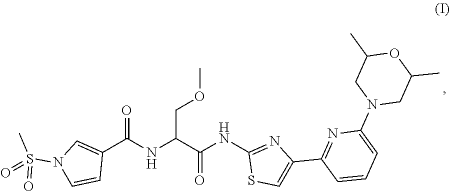 [SMILES: COCC(NC(=O)c1ccn(S(C)(=O)=O)c1)C(=O)Nc1nc(-c2cccc(N3CC(C)OC(C)C3)n2)cs1]
wherein the subject is not administered a CYP3A inhibitor, a CYP3A inducer, a sensitive CYP3A substrate with a narrow therapeutic index, a sensitive P-gp substrate with a narrow therapeutic index, a sensitive BCRP substrate with a narrow therapeutic index, or a combination thereof concomitantly with the regimen.

3. The method of any one of claim 1 or 2, wherein the subject has cancer.

4. A method of treating cancer in a subject in need thereof, the method comprising administering to the subject a therapeutic regimen comprising an effective amount of the compound of formula (I): 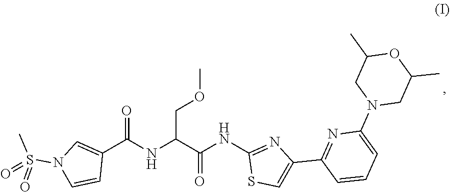 [SMILES: COCC(NC(=O)c1ccn(S(C)(=O)=O)c1)C(=O)Nc1nc(-c2cccc(N3CC(C)OC(C)C3)n2)cs1]
or a pharmaceutically acceptable salt thereof;
wherein the subject is not administered a CYP3A inhibitor, a CYP3A inducer, a sensitive CYP3A substrate with a narrow therapeutic index, a sensitive P-gp substrate with a narrow therapeutic index, a sensitive BCRP substrate with a narrow therapeutic index, or a combination thereof concomitantly with the regimen.

5. The method of claim 3 or 4, wherein the cancer is non-small cell lung cancer, colorectal cancer, bladder cancer, cancer of unknown primary, glioma, breast cancer, melanoma, non-melanoma skin cancer, endometrial cancer, esophageal cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, penile cancer, bone cancer, or hematologic cancer.

6. The method of claim 5, wherein the cancer is esophageal cancer.

7. The method of claim 5, wherein the cancer is non-small cell lung cancer, colorectal cancer, bladder cancer, cancer of unknown primary, glioma, breast cancer, melanoma, non-melanoma skin cancer, endometrial cancer, penile cancer, bone cancer, renal cell carcinoma, prostate cancer, or hematologic cancer.

8. The method of claim 7, wherein the cancer is non-small cell lung cancer.

9. The method of claim 7, wherein the cancer is melanoma, prostate cancer, breast cancer, bone cancer, renal cell carcinoma, or hematologic cancer.

10. The method of claim 9, wherein the cancer is melanoma.

11. The method of claim 10, wherein the melanoma is uveal melanoma, mucosal melanoma, or cutaneous melanoma.

12. The method of claim 11, wherein the melanoma is uveal melanoma.

13. The method of claim 12, wherein the uveal melanoma is metastatic uveal melanoma.

14. The method of claim 12, wherein the uveal melanoma is advanced uveal melanoma.

15. The method of claim 9, wherein the cancer is prostate cancer.

16. The method of claim 9, wherein the cancer is hematologic cancer.

17. The method of claim 9, wherein the hematologic cancer is multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, or non-Hodgkin's lymphoma.

18. The method of claim 9, wherein the hematologic cancer is acute myeloid leukemia.

19. The method of claim 9, wherein the cancer is breast cancer.

20. The method of claim 19, wherein the breast cancer is an ER positive breast cancer, an ER negative breast cancer, triple positive breast cancer, or triple negative breast cancer.

21. The method of claim 9, wherein the cancer is a bone cancer.

22. The method of claim 21, wherein the bone cancer is Ewing's sarcoma.

23. The method of claim 21, wherein the cancer is a renal cell carcinoma.

24. The method of claim 23, wherein the renal cell carcinoma is a microphthalmia transcription factor family translocation renal cell carcinoma.

25. The method of any one of claims 3 to 24, wherein the cancer is metastatic.

26. The method of any one of claims 3 to 24, wherein the cancer is advanced.

27. The method of any one of claims 3 to 26, wherein the cancer is resistant to, or failed to respond to prior treatment with, an anticancer therapy.

28. The method of claim 27, wherein the anticancer therapy is a chemotherapeutic or cytotoxic agent, immunotherapy, surgery, radiotherapy, thermotherapy, or photocoagulation, or a combination thereof.

29. The method of claim 28, wherein the anticancer therapy is a chemotherapeutic or cytotoxic agent.

30. The method of claim 29, wherein the chemotherapeutic or cytotoxic agent is a mitogen-activated protein kinase (MEK) inhibitor and/or a protein kinase C (PKC) inhibitor.

31. The method of any one of claims 3 to 30, wherein the cancer is resistant to, or failed to respond to prior treatment with a PKC inhibitor.

32. The method of any one of claims 3 to 31, wherein the method further comprises administering to the subject an anticancer therapy.

33. The method of claim 32, wherein the anticancer therapy is a chemotherapeutic or cytotoxic agent, immunotherapy, surgery, radiotherapy, thermotherapy, or photocoagulation, or a combination thereof.

34. The method of claim 32 or 33, wherein the anticancer therapy is surgery, a MEK inhibitor, and/or a PKC inhibitor, or a combination thereof.

35. The method of claim 34, wherein the MEK inhibitor is selumetinib, binimetinib, or tametinib.

36. The method of claim 34, wherein the PKC inhibitor is sotrastaurin or IDE196.

37. The method of any one of claims 1 to 36, wherein the CYP3A inhibitor is a strong CYP3A inhibitor.

38. The method of claim 37, wherein the strong CYP3A inhibitor is boceprevir, cobicistat, danoprevir, elvitegravir, grapefruit juice, indinavir, itraconazole, ketoconazole, lopinavir, paritaprevir, ombitasvir, dasabuvir, posaconazole, ritonavir, saquinavir, telaprevir, tipranavir, telithromycin, troleandomycin, voriconazole, clarithromycin, idelalisib, nefazodone, nelfinavir, or a pharmaceutically acceptable salt thereof, or a combination thereof.

39. The method of any one of claims 1 to 38, wherein the CYP3A inducer is a strong CYP3A inducer.

40. The method of claim 39, wherein the strong CYP3A inducer is apalutamide, carbamazepine, enzalutamide, mitotane, phenytoin, rifampin, St. John's wort, or a pharmaceutically acceptable salt thereof, or a combination thereof.

41. The method of any one of claims 1 to 40, wherein the sensitive CYP3A substrate with a narrow therapeutic index is alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, tolvaptan, or a pharmaceutically acceptable salt thereof, or a combination thereof.

42. The method of any one of claims 1 to 41, wherein the sensitive P-gp substrate with a narrow therapeutic index is an orally administered sensitive P-gp substrate with a narrow therapeutic index.

43. The method of any one of claims 1 to 42, wherein the sensitive P-gp substrate with a narrow therapeutic index is dabigatran etexilate, digoxin, fexofenadine, loperamide, quinidine, talinolol, vinblastine, or a pharmaceutically acceptable salt thereof, or a combination thereof.

44. The method of any one of claims 1 to 43, wherein the sensitive BCRP substrate with a narrow therapeutic index is an orally administered sensitive BCRP substrate with a narrow therapeutic index.

45. The method of any one of claims 1 to 43, wherein the sensitive BCRP substrate with a narrow therapeutic index is coumestrol, daidzein, dantrolene, estrone-3-sulfate, genistein, prazosin, sulfasalazine, rosuvastatin, or a pharmaceutically acceptable salt thereof, or a combination thereof.

46. The method of any one of claims 1 to 45, wherein the subject is not administered an acid-reducing agent concomitantly with the regimen; provided that, an acid-reducing agent that is an antacid may be concomitantly administered with the therapeutic regimen in a staggered dosing manner.

47. The method of claim 46, wherein the acid-reducing agent is an antacid, H2 blocker, proton pump inhibitor, or a combination thereof.

48. The method of claim 47, wherein the H2 blocker is famotidine, cimetidine, ranitidine, nizatidine, or a combination thereof.

49. The method of claim 47 or 48, wherein the proton pump inhibitor is omeprazole, lansoprazole, pantoprazole, rabeprazole, esomeprazole, dexlansoprazole, ilaprazole, or a combination thereof.

50. The method of any one of claims 1 to 49, wherein the compound of formula (I) is administered orally.

51. The method of any one of claims 1 to 50, wherein the compound of formula (I) is administered in a unit dosage form selected from the group consisting of capsule or tablet.

52. The method of any one of claims 1 to 51, wherein the compound of formula (I) has the following structure:

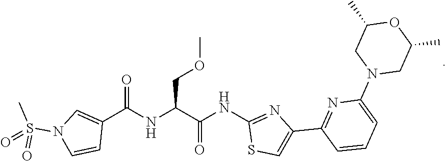 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

53. The method of any one of claims 1 to 52, wherein the compound of formula (I) is administered as a pharmaceutical composition comprising the compound of formula (I) or a pharmaceutically acceptable salt thereof.

54. The method of claim 53, wherein the pharmaceutical composition comprises one or more of a filler, a disintegrant, a wetting agent, a glidant, a lubricant, and a capsule shell.

55. The method of claim 54, wherein the filler is microcrystalline cellulose, mannitol, or a combination thereof.

56. The method of claim 54 or 55, wherein the pharmaceutical composition comprises 70 to 90% (w/w) of the filler.

57. The method of any one of claims 54 to 56, wherein the disintegrant is croscarmellose sodium.

58. The method of any one of claims 54 to 57, wherein the pharmaceutical composition comprises 4 to 6% (w/w) of the disintegrant.

59. The method of any one of claims 54 to 58, wherein the wetting agent is sodium lauryl sulfate.

60. The method of any one of claims 54 to 59, wherein the pharmaceutical composition comprises 0.5 to 1.5% (w/w) of the wetting agent.

61. The method of any one of claims 54 to 60, wherein the glidant is colloidal silicon dioxide

62. The method of any one of claims 54 to 61, wherein the pharmaceutical composition comprises 1.5 to 2.5% (w/w) of the glidant.

63. The method of any one of claims 54 to 62, wherein the lubricant is magnesium stearate.

64. The method of any one of claims 54 to 63, wherein the pharmaceutical composition comprises 0.4 to 0.6% (w/w) of the lubricant.

65. The method of any one of claims 54 to 64, wherein the pharmaceutical composition comprises a capsule shell comprising a polymeric shell.

66. The method of claim 65, wherein the polymeric shell comprises hypromellose and titanium dioxide.

67. The method of any one of claims 54 to 66, wherein the pharmaceutical composition is a unit dosage form.

68. The method claim 67, wherein the unit dosage form is a capsule.

69. The method of any one of claims 53 to 68, wherein the pharmaceutical composition comprises 2.5 to 20% (w/w) of the compound of formula (I) ora pharmaceutically acceptable salt thereof.

70. The method of claim 53, wherein the pharmaceutical composition comprises:
2.5 to 20% (w/w) of the compound of formula (I) or a pharmaceutically acceptable salt thereof;
70 to 90% (w/w) of the filler;
4 to 6% (w/w) of the disintegrant;
0.5 to 1.5% (w/w) of the wetting agent;
1.5 to 2.5% (w/w) of the glidant; and
0.4 to 0.6% (w/w) of the lubricant.

71. The method of claim 53, wherein the pharmaceutical composition comprises:
2.5 to 20% (w/w) of the compound of formula (I) or a pharmaceutically acceptable salt thereof;
70 to 90% (w/w) of microcrystalline cellulose, mannitol, or a combination thereof;
4 to 6% (w/w) of croscarmellose sodium;
0.5 to 1.5% (w/w) of sodium lauryl sulfate;
1.5 to 2.5% (w/w) of colloidal silicon dioxide; and
0.4 to 0.6% (w/w) of magnesium stearate.

72. The method of any one of claims 53 to 71, wherein the pharmaceutical composition comprises 2.5 mg to 20 mg of the compound of formula (I) ora pharmaceutically acceptable salt thereof.

73. The method of claim 53, wherein the pharmaceutical composition comprises:
2.6% (w/w) of the compound of formula (I) ora pharmaceutically acceptable salt thereof;
50.8% (w/w) of microcrystalline cellulose;
38.1% (w/w) of mannitol;
5% (w/w) of croscarmellose sodium;
1.0% (w/w) of sodium lauryl sulfate;
2.0% (w/w) of colloidal silicon dioxide; and
0.5% (w/w) of magnesium stearate.

75. The method of claim 53, wherein the pharmaceutical composition comprises:
20% (w/w) of the compound of formula (I) or a pharmaceutically acceptable salt thereof;
48.2% (w/w) of microcrystalline cellulose;
37.0% (w/w) of mannitol;
5.8% (w/w) of croscarmellose sodium;
1.2% (w/w) of sodium lauryl sulfate;
2.3% (w/w) of colloidal silicon dioxide; and
0.6% (w/w) of magnesium stearate.

76. The method of any one of claims 53 to 74, wherein the pharmaceutical composition comprises 2.5 mg of the compound of formula (I) or a pharmaceutically acceptable salt thereof.

77. The method of any one of claims 53 to 73 and 75, wherein the pharmaceutical composition comprises 20 mg of the compound of formula (I) or a pharmaceutically acceptable salt thereof.

78. The method any one of claims 70 to 77, wherein the pharmaceutical composition is a unit dosage form that is a capsule.

79. The method of claim 78, wherein the capsule comprises a capsule shell comprising a polymeric shell. dioxide.

80. The method of claim 79, wherein the polymeric shell comprises hypromellose and titanium dioxide.
